(19) United States
(12) Patent Application Publication
(10) Pub. No.: US 2022/0401479 A1
(43) Pub. Date: Dec. 22, 2022

(54) LARGE-SCALE COMBINED CAR TRANSDUCTION AND CRISPR GENE EDITING OF T CELLS

(71) Applicant: BOARD OF REGENTS, THE UNIVERSITY OF TEXAS SYSTEM, Austin, TX (US)

(72) Inventors: Katy REZVANI, Houston, TX (US); Rafet BASAR, Houston, TX (US); Elif GOKDEMIR, Houston, TX (US); Mayra SHANLEY, Houston, TX (US)

(21) Appl. No.: 17/756,078

(22) PCT Filed: Nov. 25, 2020
(86) PCT No.: PCT/US2020/062324

§ 371 Date: May 16, 2022

Related U.S. Application Data
(60) Provisional application No. 62/941,638, filed on Nov. 27, 2019.
(60) Provisional application No. 63/022,828, filed on May 11, 2020.

(51) Int. Cl. A61K 35/17; C12N 5/0783; C12N 15/11; C12N 9/22; C12N 15/62; C12N 15/90; A61K 38/17; A61P 31/12

(52) U.S. Cl. CPC A61K 35/17; C12N 5/0636; C12N 15/11; C12N 9/22; C12N 15/625; C12N 15/907; A61K 38/1774; A61P 31/12; C12N 2310/20; C12N 2800/80; C12N 2501/2312; C12N 2501/2318; C12N 2501/2321; C12N 2500/70

(57) ABSTRACT

Embodiments of the disclosure encompass methods and compositions for producing engineered T cells. The disclosure concerns large-scale processes for producing T cells that may be engineered to have disruption of expression of one or more genes using CRISPR and also express at least one heterologous antigen receptor. Specific embodiments include particular parameters for the process. The T cells may or may not be viral-specific.

This application claims priority to U.S. Provisional Patent Application Ser. No. 62/941,638, filed Nov. 27, 2019, and claims priority to U.S. Provisional Patent Application Ser. No. 63/022,828, filed May 11, 2020, both of which applications are incorporated by reference herein in their entirety.

### SEQUENCE LISTING

The instant application contains a Sequence Listing which has been submitted electronically in ASCII format and is hereby incorporated by reference in its entirety. Said ASCII copy, created on Nov. 13, 2020, is named UTSC_P1201WO_SL.txt and is 2,236 bytes in size.

### TECHNICAL FIELD

The present disclosure relates generally to the fields of immunology, cell biology, molecular biology, cell therapy, and medicine.

### BACKGROUND

Cellular immunotherapy holds much promise for the treatment of cancer. However, most immunotherapeutic approaches when applied alone are of limited value against the majority of malignancies, especially solid tumors. Reasons for this limited success include (1) reduced expression of tumor antigens on the surface of tumor cells, which reduces their detection by the immune system; (2) the expression of ligands for inhibitory receptors, such as PD1, NKG2A, TIGIT; (3) upregulation of cellular checkpoints, such as CISH, that induce immune cell inactivation; and (4) the induction of cells (e.g., regulatory T cells or myeloid-derived suppressor cells) in the microenvironment that release substances such as transforming growth factor-β (TGFβ) and adenosine that suppress the immune response and promote tumor cell proliferation and survival. Thus, there is an unmet need for improved methods of cellular immunotherapy, including T cells.

### SUMMARY

Embodiments of the disclosure include methods and compositions that enhance use of adoptive cell therapy to treat a medical condition, such as cancer. In particular embodiments, the adoptive cell therapy comprises T cells that have been specifically engineered to have enhancement of efficacy compared to T cells that have not been so engineered. In specific embodiments, the T cells are engineered to allow them to avoid or overcome suppression of immune responses in individuals that are recipients of the T cells as part of a therapy, such as for cancer. In particular embodiments, the engineered T cells overcome natural processes in recipient individuals that promote tumor cell proliferation and survival of cancer cells. The engineered T cells have enhanced activity against cancer cells compared to T cells lacking the engineering. The T cells are engineered by the hand of man to have enhanced anti-cancer activity, as opposed to having enhanced activity because of natural mutations, for example.

Embodiments of the disclosure encompass novel, large-scale approaches (including GMP grade) for the combined CAR transduction and CRISPR gene editing of T cells (which includes T cells of any kind, such as cytotoxic CD8+ T lymphocyte (CTL) cells, CD4+ T cells, regulatory T cells, gamma delta T cells, tumor-infiltrating T cells (TILs) and mixtures thereof). The disclosure provides for production methods, methods of use, and compositions directed to engineered T cells of any kind, including CTL cells, that express one or more heterologous antigen receptors and/or have been gene edited to have disruption of one or more endogenous genes in the T cells. The T cells may or may not be engineered to be viral-specific. The T cells may or may not have been positively or negatively selected for viral specificity or expanded with viral antigen to skew their specificity. The T cells may be specific towards one or more than one virus. In some embodiments, T cells, including CTLs, are produced having one or more knocked out genes and/or one or more chimeric antigen receptors (CARs), one or more T cell receptors (TCR) (e.g., engineered by the hand of man as opposed to endogenous to the cell), or combinations thereof. In specific embodiments, heterologous antigen receptor-transduced T cells are engineered to have disruption of expression of one or more endogenous genes in the T cells, and in other embodiments T cells having disruption of expression of one or more endogenous genes in the T cells are transduced or transfected to express one or more heterologous antigen receptors. Although the gene editing may be by any mechanism, in specific embodiments gene editing occurs through CRISPR techniques. Particular embodiments provide for large-scale CRISPR/Cas9-mediated engineering strategies of T cells, including, for example, virus-specific T cells and/or primary CAR-transduced T cells. In any methods and compositions, the T cells may be engineered to express one or more heterologous cytokines.

In particular embodiments, the gene that has disruption of expression in the T cells is an inhibitory gene, such as an inhibitory gene selected from the group consisting of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, TDAG8, CD5, CD7, SLAMF7, CD38, LAG3, TCR, beta2-microglobulin, HLA, CD73, CD39 and a combination thereof.

In particular embodiments, the gene that has disruption of expression in the T cells is NR3C1, which encodes for the glucocorticoid receptor. In some embodiments, the expression of NR3C1 is disrupted by CRISPR/Cas-9 mediated genetic engineering, although it may be disrupted by other methods. In some embodiments, the guide RNAs used to disrupt the expression of NR3C1 comprise the sequence of TGAGAAGCGACAGCCAGTGA (SEQ ID NO:1) and/or the sequence of GGCCAGACTGGCACCAACGG (SEQ ID NO:2), although these are merely examples. T cells with a disruption of NR3C1 expression may be resistant to lymphocytotoxic effects of glucocorticoids while retaining their phenotype, function, and/or specificity. As such, these cells as therapeutic compositions themselves and lacking the glucocorticoid receptor are useful to circumvent inhibition of glucocorticoids when administered to avoid deleterious immune responses in a recipient, such as following hematopoietic stem cell transplant (HSCT) in the recipient. In some embodiments, such T cells are administered to an individual with or without glucocorticoids. The administration of the T cells with glucocorticoids may reduce complications, such as graft-vs-host disease, while retaining the function of the administered T cells, in particular embodiments. Examples of glucocorticoids include at least beclomethasone, betamethasone, budesonide cortisone, dexamethasone, hydrocortisone, methylprednisolone, prednisolone, prednisone, triamcinolone, or a combination thereof. Thus, embodiments of the disclosure include methods of treating a medical condition in an individual in need of glucocorticoids, comprising the step of administering to the individual glucocorticoids and T cells engineered to have disruption of expression of the glucocorticoid receptor. In such methods, the T cells may also be engineered for one or more other characteristics, as encompassed herein.

The methods of the disclosure allow for generating primary human T-cells from any source (peripheral blood, cord blood, tumor infiltrating lymphocytes, bone marrow, cell lines, or a mixture thereof) with CRISPR/Cas9 editing, at least. The T cells from these sources may be alternatively engineered or additionally engineered for one or more other characteristics, as encompassed herein.

In certain embodiments, a process for producing engineered T cells utilizes specific parameters that allow the process to be large-scale, including for engineering up to 1×10⁹, 1×10¹⁰, 1×10¹¹, or more cells. In particular embodiments, the process utilizes successive steps including one or multiple expansion steps; optionally one or more depletion steps to remove cells positive for particular marker(s) (thereby removing undesired cells that express said marker(s)); modification of the cells to allow the T cells to express one or more heterologous antigen receptors; modification of the cells to lack expression, or have reduced expression, of one or more genes endogenous in the T cells; or a combination thereof. The order of the successive steps may or may not be of any order. In specific cases, one or more endogenous genes are knocked out or knocked down using gene editing techniques (such as CRISPR and guide RNAs) for multiple genes. The process also encompasses specific durations of time for particular steps of the process, in specific embodiments.

The produced cells may be used for any purpose, including for the treatment of a medical condition such as cancer, infectious disease, and/or immune-related disorders. In some embodiments, the produced cells may be used for the treatment of viral diseases, such as viral infections from cytomegalovirus (CMV), Epstein-Barr virus (EBV), adenovirus, SARS-CoV-2 (COVID-19), SARS-CoV, JC virus, HHV6, influenza, parainfluenza, RSV, HIV, and/or BK virus (BKV). Such viral infections may or may not be the result of allogeneic HSCT.

In particular embodiments, one or more compositions are delivered to any cells by electroporation. In cases wherein cells are electroporated, an electroporation may use between about 200,000 cells to 1×10⁹ T cells. An electroporation may use between about 200,000 and 2,000,000 cells. An electroporation may use between about 1,000,000 to 1×10⁹ or more T cells, in some cases.

In particular embodiments, the heterologous antigen receptor is a chimeric antigen receptor and/or a T cell receptor. The heterologous antigen receptor may target a cancer antigen, including a tumor associated antigen. In specific cases, the heterologous antigen receptor targets an antigen selected from the group consisting of CD19, CD319 (CS1), ROR1, CD20, CD70, carcinoembryonic antigen, alphafetoprotein, CA-125, MUC-1, epithelial tumor antigen, melanoma-associated antigen, mutated p53, mutated ras, HER2/Neu, ERBB2, folate binding protein, HIV-1 envelope glycoprotein gp120, HIV-1 envelope glycoprotein gp41, GD2, CD5, CD123, CD23, CD30, CD56, CD70, CD38, c-Met, mesothelin, GD3, HERV-K, IL-11Ralpha, kappa chain, lambda chain, CSPG4, ERBB2, WT-1, TRAIL/DR4, VEGFR2, CD33, CD47, CLL-1, U5snRNP200, CD200, BAFF-R, BCMA, CD99, and a combination thereof, although other antigens are encompassed herein.

Embodiments of the disclosure include populations of T cells produced by any method encompassed herein. Compositions comprising the population of cells of the disclosure are contemplated, including when the population is in a pharmaceutically acceptable carrier. The population of cells may be produced and may or may not be stored prior to use. In some cases, a population of cells produced by methods herein are stored upon completion of the preparation process, or they may be stored following an intermediate step and further modified at a later date. In some cases, the T cells are stored following production and following storage but prior to use they are further modified; such modification can be addition of one or more heterologous antigen receptors; disruption of expression of one or more endogenous genes; rendering the T cells to be viral-specific, and so forth. In some cases, the T cells have disruption of expression of one or more endogenous genes and are then stored. Prior to use the T cells may be further modified to express a heterologous antigen receptor that targets an antigen on cancer cells of an individual in need thereof and/or the T cells may be further modified to be viral-specific for a virus in an individual in need of treatment for the virus.

Embodiments of the disclosure include methods of treating an individual for a medical condition, comprising the step of administering to the individual a therapeutically effective amount of T cells produced by any method of the disclosure.

Other objects, features and advantages of the present disclosure will become apparent from the following detailed description. It should be understood, however, that the detailed description and the specific examples, while indicating particular embodiments of the disclosure, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D show CRISPR-Cas9 mediated deletion of the gene coding for the glucocorticoid receptor (GR) in primary human VST cells. FIG. 1A, Schematic summary of the protocol for CRISPR-Cas9-mediated knockout (KO) of NR3C1 in primary human virus-specific T cells (VSTs). Peripheral blood mononuclear cell (PBMC) were stimulated with virus-specific pepMixes from CMV, BKV and adenovirus (in combination) in the presence of IL-7 10 ng/ml, IL-2 50 IU/ml and IL-15 10 ng/ml. On days 7-10 of in vitro expansion, primary human VSTs were nucleofected with control Cas9 alone (Cas9 control) or Cas9 preloaded with guide RNA (gRNA) targeting the exon 2 of the NR3C1 gene, which encodes for the GR protein. KO efficiency and functional assays were performed at day +14 post initial PBMC isolation. FIG. 1B, Schematic representation of CRISPR-Cas9 mediated NR3C1 KO using two short guide CRISPR RNAs (crRNAs) targeting the exon 2 of NR3C1 gene. The crRNA1 sequences in FIG. 1B are in a 5′ to 3′ direction CCTTGAGAAGCGACAGCCAGTGA (SEQ ID NO:5) and its complement in a 5′ to 3′ direction is TCACTGGCTGTCGGCTTCTCAAGG (SEQ ID NO:6). The crRNA2 sequences in 1B are in a 5′ to 3′ direction are CCTGGCCAGACTGGCACCAACGG (SEQ ID NO:7) and its complement in a 5′ to 3′ direction is CCGTTGGTGCCAGTCTGGCCAGG (SEQ ID NO:8). FIGS. 1C-1D, The NR3C1 KO efficiency of VST cells after electroporation with Cas9 alone (control), Cas9 complexed with one crRNA (crRNA 1 or crRNA 2) or Cas9 complexed with the combination of two crRNA's (crRNA 1+crRNA 2) using a wild type Cas9 was determined by PCR analysis at day 3 (FIG. 1C) or western blot analysis at day 7 (FIG. 1D) after electroporation.

FIGS. 2A-2F show NR3C1 deletion renders VST cells resistant to steroids without altering their phenotype or function. FIG. 2A, Representative FACS plots showing the percentage of apoptotic cells (Annexin-V+) and alive or dead cells (Live/Dead stain) in control Cas9 vs NR3C1 KO (exon 2) VST cells after culture with or without Dexamethasone (Dexa; 200 μM) for 72 hours (n=4). Inset values indicate the percentage of Annexin-V and alive/dead cells from each group. FIGS. 2B, 2C, Graph summarizing the percentage of live cells (FIG. 2B) and the absolute cell number (FIG. 2C) between control Cas9 and NR3C1 KO VST cells treated with or without 200 μM of Dexamethasone for 72 hours (n=4). Statistical significance is indicated as **p≤0.01; NS, not significant. Bars represent mean values with standard deviation. FIG. 2D, FACS plots showing the frequency of CD62L and CCR7 in control Cas9 or NR3C1 KO VST cells that were treated with or without Dexa (200 μM) for 72 hours (n=3). Inset values indicate the percentage of T-cells expressing CD62L and/or CCR7 in each treatment group. These FACS plots were pre-gated on CD3+CD45RA− T cells. FIGS. 2E, 2F, Bar graphs showing the percentage of IFN-γ, TNF-α or IL-2 production by CD8+ (FIG. 2E) and CD4+ (2F) VSTs treated with control Cas9 (black; left bar of groupings of three), NR3C1 KO (blue; middle bar of groupings of three) or NR3C1 KO+Dexamethasone (Dexa; 200 μM; red; right bar of groupings of three) in response to 6 hrs of stimulation with viral pepmix (n=3). The functional analysis of the Cas9+Dexa group was not performed due to the absence of viable cells resulting from the lymphocytotoxic effect of steroids. The bars represent mean values with standard deviation. NS, not significant.

FIGS. 3A-3C show NR3C1 KO VST cells persist in vivo even after treatment with dexamethasone. FIG. 3A, Schematic diagram representing the timeline for the in vivo experiments. NSG mice received one dose (1×106) of control Cas9 or NR3C1 KO VST cells and were either observed (top panel) or treated with daily subcutaneous dose of dexamethasone (15 mg/kg) for 2 weeks (bottom panel) (n=5 mice/group). FIG. 3B, All mice were sacrificed on day +14 after the adoptive infusion of Cas9 control or NR3C1 KO VST cells. Cells were collected from the bone marrows and analyzed by flow cytometry for the expression of human (h)CD45 and CD3. Representative FACS plots are presented. Inset values indicate the percentage of CD3 positive cells from each group. FIG. 3C, Bar graph shows the pooled data for the percentage of CD3 positive cells from panel (B) (n=5). The bars represent mean values with standard deviation. The statistical significance is indicated as *p≤0.05; NS, not significant.

FIGS. 4A-4C show identification of Cas9 off-target sites by GUIDE-seq and quantification of potential Cas9 off-target cleavage sites using rhAmpSeq technology. FIG. 4A, Sequences of off-target sites identified by GUIDE-seq for two guides RNA (gRNA) targeting the NR3C1 locus (gRNA #1 upper panel and gRNA #2 lower panel; TGAGAAGCGACAGCCAGTGA is SEQ ID NO:1 GGCCAGACTGGCACCAACGG is SEQ ID NO:2). The guide sequence is listed on top with off-target sites shown below. The on-target site is identified with a black square. Mismatches to the guide are shown and highlighted in color with insertions shown in grey below. The number of GUIDE-seq sequencing reads are shown to the right of each site. Pie charts indicate the fractional percentage of the total unique, CRISPR-Cas9 specific read counts that are on-target (orange) and off-target (blue). FIG. 4B, On- and off-target effects were determined by targeted amplification followed by next-generation sequencing (NSG) for exon 2-guide 1 and for exon 2-guide 2. Individual guide RNAs were delivered into HEK293 cells stably expressing WT-Cas9 (left panels), or delivered into HEK293 cells by complexing to WT-Cas9 protein (middle panels) or HiFi-Cas9 protein (right panels). Pie charts indicate the percentage of on-target effect (in red) and off-target effect (in blue). FIG. 4C, Editing efficiency was determined using targeted amplification followed by next-generation sequencing. Exon 2-Guide 1 and Exon 2-Guide 2 guide RNAs were complexed simultaneously with either WT-Cas9 (blue bars on the left) or HiFi-Cas9 (orange bars on the right) into HEK293 cells. Editing efficiencies were determined for the known on- and off target sites for Exon 2-Guide 1 (left upper panel), and Exon 2-Guide 2 (right upper panel). In a similar experiment, Exon 2-Guide 1 and Exon 2-Guide 2 guide RNAs were complexed simultaneously with either WT-Cas9 (blue bars) or HiFi-Cas9 (orange bars) into primary human T-cells. Editing efficiencies were determined for the known on- and off target sites for Exon 2-Guide 1 (left lower panel), and Exon 2-Guide 2 (right lower panel). Data are shown as mean and standard deviation from n=3 human donors.

FIGS. 5A-5F show successful scale-up of CRISPR-Cas9-mediated NR3C1 deletion in VST cells. FIG. 5A, Different VST cell numbers (3×106, 25×106 and 100×106) were electroporated in the presence of Cas9 and gRNA using the Lonza 4D nucleofector and the NR3C1 KO efficiency in Cas9 (control) or NR3C1 KO (KO) VST cells was determined using PCR (FIG. 5A, n=3) and western blot analysis (FIG. 5B, n=3). β-actin was used as loading control in panel (FIG. 5B). FIG. 5C, Representative FACS plots showing the percentage of apoptotic cells (Annexin-V+) and alive or dead cells (Live/Dead stain) in control Cas9 and NR3C1 KO VST cells at the different cell dose levels of 3×10⁶, 25×106 or 100×10⁶ cells/electroporation treated with or without Dexa (200 μM) for 72 hours (n=3). FIG. 5D, Graph summarizing the absolute cell number between control Cas9 and NR3C1 KO VST cells at the different cell dose levels of 3×10⁶, 25×10⁶ or 100×10⁶ cells/electroporation treated with or without dexamethasome (Dexa; 200 μM) for 72 hours (n=3). Bars represent mean values with standard deviation. 5E, 5F, Bar graphs showing the percentage of IFN-γ, TNF-α or IL-2 production by 100×10⁶ control Cas9 (black; left in the groupings of three), 100×106 NR3C1 KO (blue; middle in the groupings of three) or 100×106 NR3C1 KO+Dexamethasone (Dexa 200 μM; red; right in the groupings of three) VST cells in response to 6 hour stimulation with the relevant viral pepmix in the CD8+ T cell (FIG. 5E) and CD4+ T cell (FIG. 5F) compartments (n=3). The functional analysis of the Cas9+Dexa group was not performed due to the absence of viable cells resulting from the lymphocytotoxic effect of steroids. The bars represent mean values with standard deviation. NS, not significant.

FIGS. 6A-6B show functional studies of multivirus specific VSTs compared to single-specificity VSTs. FIG. 6A, Representative FACS plots showing the percent of cytokine production (IFN-γ, TNF-α or IL-2) in multivirus specific VSTs Cas9 control or NR3C1 KO in response to stimulation with individual virus pepmix (CMV, BKV or ADV) in the CD4+ T cell compartment (n=3). These FACS plots were gated on CD3+ T cells. The CD8 compartment is represented as CD4− T cell population. FIG. 6B, Representative FACS plots showing the percentage of cytokine production (IFN-γ, TNF-α or IL-2) in VSTs targeting individual viruses in response to stimulation with CMV, BKV or ADV pepmix in the CD4 and CD8 compartments (n=3).

FIGS. 7A-7F show no evidence for graft-versus-host-disease (GVHD) or toxicity in NSG mice receiving Cas9 control or NR3C1 KO VST cells. NSG mice infused with one dose of 1×106 Cas9 control or NR3C1 KO VST cells and treated with or without daily subcutaneous dose of dexamethasone (15 mg/kg) were sacrificed 2 weeks after infusion and examined for any evidence of toxicity as well as lesions attributable to graft-versus-host disease (GVHD) including necrosis with lymphocytic infiltrate. Representative micrographs of lungs (FIG. 7A), liver (FIG. 7B), kidney (FIG. 7C), small intestine (FIG. 7D), skin (FIG. 7E) and brain (FIG. 7F) show no evidence of pathology. Minimal lymphocytic infiltration of portal areas in the liver was observed in control mice that received Cas9 control VSTs and not treated with glucocorticoids. H&E staining magnification 5× (left), magnification 20× (right).

FIGS. 8A-8B shows efficient CRISPR-Cas9 deletion of the NR3C1 gene in VST cells using a High Fidelity (HIFI) Cas9 protein. FIG. 8A, B The NR3C1 KO efficiency of VST cells after electroporation with Cas9 alone (control), Cas9 complexed with one crRNA (crRNA 1 or crRNA 2) or Cas9 complexed with the combination of two crRNA's (crRNA 1+crRNA 2) using a High Fidelity (HiFi) Cas9 was determined by PCR analysis at day 3 (FIG. 8A) or western blot analysis at day 7 (FIG. 8B) after electroporation.

FIG. 9 shows frequencies of CD4 and CD8 T-cells in NR3C1 KO VSTs generated using a large scale GMP protocol. Representative FACS plots show the percentage of CD4 and CD8 T cells in Cas9 control VSTs or NR3C1 KO VSTs at each scale up dose treated before and after treatment with dexamethasone (Dexa; 200 μM) for 72 hours. Of note, no FACS plot is represented for Cas9 control VSTs after dexamethasone treatment due to the absence of viable cells in that group.

FIG. 10 shows one embodiment for large scale GMP production of CRISPR-Cas9-mediated NR3C1 KO VSTs for the treatment of viral infections in immunosuppressed patients. Schematic diagram representing the steps involved in the production, expansion and biobanking of the CRISPR-Cas9-mediated NR3C1 KO VSTs using GMP grade materials and equipment. This figure was generated using Biorender.

FIGS. 11A-11B demonstrate NR3C1 knockout efficiency being tested using PCR for various programs (CM137, DN100, CA137, DS137 and EH100) for electroporation in Lonza 4D-Nucleofactor™. This was performed in small scale, with 5E6 T cells and Plasmalyte (supplemented with HEPES and mannitol, PL); FIG. 11A shows knockout of the NR3C1 gene in the T cells using the various manufacturer programs. FIG. 11B shows fold expansion of NR3C1 knockout cells tested with various electroporation programs.

FIGS. 12A-12B concern NR3C1 knockout efficiency using PCR tested for the CM137 program used for electroporation in Lonza 4D-Nucleofactor™. This was done in large scale, with 50E6, using either Lonza buffer (P3) or PL (supplemented with HEPES and mannitol) and with 100E6 using PL (supplemented with HEPES and mannitol) (FIG. 12A). FIG. 12B shows NR3C1 knockout efficiency using CA137 program for electroporation in Lonza 4D-Nucleofactor™. This was done in large scale, with either 50E6 or 100E6 using PL (supplemented with HEPES and mannitol).

### DESCRIPTION OF ILLUSTRATIVE EMBODIMENTS

### I. Definitions

As used herein the specification, “a” or “an” may mean one or more. As used herein in the claim(s), when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. In specific embodiments, aspects of the disclosure may “consist essentially of” or “consist of” one or more sequences of the disclosure, for example. Some embodiments of the invention may consist of or consist essentially of one or more elements, method steps, and/or methods of the disclosure. It is contemplated that any method or composition described herein can be implemented with respect to any other method or composition described herein. The scope of the present application is not intended to be limited to the particular embodiments of the process, machine, manufacture, composition of matter, means, methods and steps described in the specification. As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. The terms “about”, “substantially” and “approximately” mean, in general, the stated value plus or minus 5%.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

An “immune disorder,” “immune-related disorder,” or “immune-mediated disorder” refers to a disorder in which the immune response plays a key role in the development or progression of the disease Immune-mediated disorders include autoimmune disorders, allograft rejection, graft versus host disease and inflammatory and allergic conditions.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. With respect to cells, the cells may be engineered because they have reduced expression of one or more endogenous genes and/or because they express one or more heterologous genes (such as synthetic antigen receptors and/or cytokines), in which case(s) the engineering is all performed by the hand of man With respect to an antigen receptor, the antigen receptor may be considered engineered because it comprises multiple components that are genetically recombined to be configured in a manner that is not found in nature, such as in the form of a fusion protein of components not found in nature so configured.

The term “large-scale” as used herein refers to on the order of up to 10⁹ or more, including 10¹⁰, 10¹¹, and so forth number of cells.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” or “individual” refer to either a human or non-human, such as primates, mammals, and vertebrates. In particular embodiments, the subject is a human

As used herein, a “mammal” is an appropriate subject for the method of the present invention. A mammal may be any member of the higher vertebrate class Mammalia, including humans; characterized by live birth, body hair, and mammary glands in the female that secrete milk for feeding the young. Additionally, mammals are characterized by their ability to maintain a constant body temperature despite changing climatic conditions. Examples of mammals are humans, cats, dogs, cows, mice, rats, horses, goats, sheep, and chimpanzees. Mammals may be referred to as “patients” or “subjects” or “individuals”.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

As used herein, a “disruption” of a gene refers to the elimination or reduction of expression of one or more gene products encoded by the subject gene in a cell, compared to the level of expression of the gene product in the absence of the disruption. Exemplary gene products include mRNA and protein products encoded by the gene. Disruption in some cases is transient or reversible and in other cases is permanent. Disruption in some cases is of a functional or full length protein or mRNA, despite the fact that a truncated or non-functional product may be produced. In some embodiments herein, gene activity or function, as opposed to expression, is disrupted. Gene disruption is generally induced by artificial methods, i.e., by addition or introduction of a compound, molecule, complex, or composition, and/or by disruption of nucleic acid of or associated with the gene, such as at the DNA level. Exemplary methods for gene disruption include gene silencing, knockdown, knockout, and/or gene disruption techniques, such as gene editing. Examples include antisense technology, such as RNAi, siRNA, shRNA, and/or ribozymes, which generally result in transient reduction of expression, as well as gene editing techniques which result in targeted gene inactivation or disruption, e.g., by induction of breaks and/or homologous recombination. Examples include insertions, mutations, and deletions. The disruptions typically result in the repression and/or complete absence of expression of a normal or “wild type” product encoded by the gene. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

The term “heterologous” as used herein refers to being derived from a different cell type or a different species than the recipient. In specific cases, it refers to a gene or protein that is synthetic and/or not from an T cell. The term also refers to synthetically derived genes or gene constructs. In specific cases, it refers to a gene or protein that is synthetic and/or not from a T cell. The term also refers to synthetically derived genes or gene constructs. For example, a cytokine may be considered heterologous with respect to a T cell even if the cytokine is naturally produced by the T cell because it was synthetically derived, such as by genetic recombination, including provided to the T cell in a vector that harbors nucleic acid sequence that encodes the cytokine.

The present disclosure concerns a novel approach for the large-scale expansion, CAR transduction, and gene editing of T cells (which may also be referred to herein as T lymphocytes or CTLs). This approach allows expansion of T cells of any kind to be expanded to large numbers and transduced to redirect their specificity against tumor antigens, in addition to optionally expressing one or more heterologous cytokine genes. The function of T cells is further improved by deleting gene(s) involved in certain immune responses, cell exhaustion and tumor-induced dysfunction, as some examples.

In particular embodiments, the disclosure describes particular methods for combined CAR transduction and deletion of single or multiple endogenous genes in T cells; this contributes to improved function. In specific cases, the improved function includes resistance to glucocorticoids and/or resistance to the tumor microenvironment for the engineered T cells. In particular, a large-scale and GMP grade protocol is used for this approach for translation to a clinic. The disclosure has direct implications on patient care using a novel immunotherapeutic approach that enhances the function of a patient's own T cells or adoptively transferred T cells.

### II. Process

Embodiments of the disclosure concern processes of generating engineered T cells, particularly on a large scale. In specific embodiments, the process utilizes one or a combination of particular parameters to produce certain types of engineered T cells, where in at least some cases the parameters include certain concentrations of reagents, certain types of T cells, certain durations of time for one or more steps, certain types of cell modification mechanisms, or a combination thereof, as examples. The skilled artisan will recognize that although optimum variables are described herein, there are also variations of the process that will still produce a suitable amount of effective engineered T cells, and these are also encompassed herein.

The process generally concerns a succession of steps, and in specific embodiments the succession is production of T cells, engineering of the T cells in one, two or more aspects, and optional expansion of the produced engineered T cells (once or at multiple steps), followed by an optional analysis step and/or an optional administration step to an individual. In specific embodiments, the engineering includes one or more of (1) modifying the cells to express one or more heterologous proteins, such as one or more antigen receptors of any kind and/or one or more heterologous cytokines, (2) modifying the cells to have reduced expression (knockdown) or elimination of expression (knockout) of one or more endogenous genes in the T cells; and (3) optionally modifying the T cells to be virus-specific or expanding them with the relevant antigen(s) to skew their specificity. The steps may be in any order. Although the modifying of the T cells to have reduced expression or eliminated expression may occur by any means, in particular embodiments the modifying is by CRISPR.

One step for the process may be the expansion (that may also be referred to as stimulation) of T cells that allows an increase in number of T cells for eventual modification. The T cells may be obtained from a fresh source or from storage or commercially, for example. Although the T cells may be of any kind, in specific embodiments the T cells are derived from cord blood, peripheral blood, bone marrow, tumor-infiltrating lymphocytes, bone marrow, or a mixture thereof. In particular embodiments, the starting culture of T cells for the expansion step includes at least 5-100 million cells, and in some cases 1×10⁹ cells or more (such as 10¹⁰ or 10¹¹) are used. Thus, in specific cases the number of T cells for initiating the protocol is 5, 10, 15, 20, 25, 30, 35, 40, 45, 50, 55, 60, 65, 70, 75, 80, 85, 90, 95, 100 million or more T cells. An example of a range of cells may be utilized at any step of the process, including 5-100, 5-90, 5-75, 5-50, 5-25, 5-10, 10-100, 10-90, 10-75, 10-60, 10-50, 10-25, 25-100, 25-90, 25-75, 25-50, 50-100, 50-90, 50-75, 75-100, 75-90, or 90-100 million cells.

In particular embodiments, prior to initiation of the expansion step in culture, the T cells may be subject to depletion of particular undesirable cells that may be present with a population of cells in which they are included, such as B cells, monocytes, NK cells, and/or myeloid cells. The depletion step may exploit the presence of one or more certain markers on the undesirable cells as a means to cull them from the population of T cells. As one example of a means to do this, the cells may be subject to depletion using immunomagnetic beads (having antibodies to the marker on the undesirable cells attached thereto), thereby producing an enriched population of T cells. In one embodiment, T cells may be negatively selected from a collection of cells by using T cell isolation methods, such as using commercially available products. In a specific embodiment, a mixture of cells comprising T cells and undesired cells that are not T cells are subjected to one or more antibodies that target the undesired cells. As one commercially available example, T cells may be negatively selected using the T cell isolation kit from Miltenyi Biotec (Bergisch Gladbach, Germany) As one example, the T cells are exposed to a cocktail that contains antibodies against CD14 to deplete monocytes, CD15 to deplete myeloid cells, CD16 to deplete NK cells and granulocyes, CD19 to deplete B cells, CD34 to deplete stem cells, CD36 to deplete platelets, CD56 to deplete NK cells, CD123 to deplete myelod blasts, CD235a (Glycophorin A) to deplete red blood cells, and/or antibodies against one or more of CD3, CD4, and CD8.

The expansion step may or may not begin with the enriched population of T cells being counted to provide an accurate assessment of quantity for culturing of the T cells. In particular embodiments, the enriched T cells are subject to anti-CD3/CD28 beads in the presence of one or more cytokines for a particular period of time. The period of time may be at least 24, 25, 26, 27, 28, 30, 31, 32, 33, 34, 35, 36, 37, 38, 39, 40, or more hours. In specific embodiments, the period of time is from 24-40 hours, 28-28 hours, 30-36 hours, and so forth. After this period, the stimulated T cells may be subject to engineering of any kind, including for knockout/knockdown of gene expression and/or heterologous antigen receptor engineering and/or manipulation to encode one or more heterologous cytokines and/or to engineer the T cells to be viral-specific.

In specific embodiments, the expansion step(s) utilize one or more particular cytokines that may be dependent upon the type of T cell being produced. For example, for T cells the cytokine may be IL-2, IL-4, IL-7, IL-12, IL-15, IL-18 or IL-21 either alone or in combination. For viral-specific T cells, the cytokine(s) may be dependent upon the type of virus for which the T cells will be directed. As an example, IL-4 and IL-7 may be used for CMV; IL-2 and IL-7 and IL-15 may be used for JCV and BKV; IL4 and IL7 may be used for adenovirus, and IL4 and IL7 may be used for EBV; in some cases IL21, IL-18, and/or IL2 are utilized. In cases wherein T cells are multivirus-specific, the cytokines may be combined. In any method for any type of cell, the media may be changed one or more times in the presence of cytokines; the new media may or may not comprise the same composition of media, including the same cytokine(s). In specific embodiments, the concentration of cytokine(s) in the media is in the range of 100-300 units/mL, including 100, 125, 150, 175, 200, 225, 250, 275, or 300 units/mL or 1-500 nM. The expansion step may occur at a certain temperature, such as about 35° C.-38° C., including 35° C., 36° C., 37° C., or 38° C. The expansion step may occur at a certain level of oxygen, such as from 3%-7% O₂; in specific embodiments the expansion step occurs at 3%, 4%, 5%, 6%, or 7% CO₂. The expansion step may last for a particular duration of time, such as a certain number of days. In specific embodiments, the expansion step lasts for 2, 3, 4, 5, 6, 7, or more days, but in specific cases the expansion step lasts from 2-7, 2-6, 2-5, 2-4, 2-3, 3-7, 3-6, 3-5, 3-4, 4-7, 4-6, 4-5, 5-6, 5-7, or 6-7 days. In particular embodiments, the media in the expansion step may or may not be changed during the expansion, but in specific embodiments every other day of the expansion step, the media is changed. In specific cases the cells are centrifuged and resuspended in the same or a similar media than before, including with a media comprising 100, 125, 150, 175, 200, 225, 250, 275, or 300 units/mL of cytokine(s). In specific aspects, the expansion step occurs in a bioreactor, such as a gas permeable bioreactor, for example G-Rex®100M or G-Rex100®. In some aspects, any stimulating is performed in a particular amount of media, such as 3-5 L of media, such as 3, 3.5, 4, 4.5, or 5 L.

Following a certain number of days after initiation of the expansion step, the cells may be further subjected to a second negative selection (depletion step) where undesirable cells are excluded (such as B cells, NK cells, and/or myeloid/monocyte cells), although in alternative embodiments they are not subjected to a second depletion step. In specific cases, on 3, 4, 5, 6, 7, 8, 9, 10, or more days after initiation of the expansion step, the cells are subjected to a depletion step.

On about days 3, 4, 5, 6, 7 or 8 following initiation of expansion of the T cells, the expanded T cells may be subjected to modification of any kind including as encompassed herein. The first modification may be transduction or transfection of the T cells to express one or more heterologous antigen receptors and/or one or more heterologous cytokines, although in some cases the first modification is disrupting expression of one or more endogenous genes of the T cells. When the first modification is transduction or transfection of the T cells to express one or more heterologous antigen receptors and/or one or more heterologous cytokines, a subsequent modification may be disrupting expression of one or more endogenous genes of the T cells, at least in some cases. When the first modification is disrupting expression of one or more endogenous genes of the T cells, a subsequent modification may be transduction or transfection of the T cells to express one or more heterologous antigen receptors and/or one or more heterologous cytokines.

In specific embodiments, the expanded T cells are transduced or transfected to harbor at least one heterologous antigen receptor gene (and/or one or more heterologous cytokine genes) prior to the cells being gene edited for disruption of expression of one or more endogenous genes. In particular embodiments, the cells are transduced or transfected with a particular vector that comprises an expression construct that encodes one or more chimeric antigen receptors, one or more T cell receptors, one or more heterologous cytokines, or a combination thereof. In other cases, the cells are transduced or transfected with multiple vectors, each comprising an expression construct and each encoding, e.g., one or more chimeric antigen receptors, one or more T cell receptors, one or more heterologous cytokines, or a combination thereof. Any vector may also encode a suicide gene. The vector may be of any kind including at least nanoparticles, plasmid, lentiviral vector, retroviral vector, adenoviral vector, adenoviral-associated vector, and so forth. The T cells may be transfected or transduced with a vector that allows for the T cells to express multiple heterologous proteins, such as a heterologous antigen receptor(s), a suicide gene, and one or more cytokines, such as one or more heterologous cytokines selected from the group consisting of IL-4, IL-10, IL-7, IL-2, IL-15, IL-12, IL-18, IL-21, and a combination thereof. Although genes for the multiple heterologous proteins may be present on the same vector, in some cases they are present on multiple vectors. Once the cells have been transduced or transfected, they are transgenic and may or may not be tested for expression of the heterologous protein(s), and this may occur 1, 2, 3, or more days following transfection/transduction, for example. When testing an aliquot from the population of transgenic T cells, the testing may or may not occur prior to further modification, such as prior to gene editing of the T cells.

Within about day 2, 3, 4, 5, 6, 7, 8, 9, 10, 11, or 12 following initiation of expansion of the T cells, the transgenic T cells harboring the heterologous antigen receptor(s) are subjected to methods of gene editing. The gene editing step may occur within 1, 2, 3, or more days following the transfection/transduction step. The gene editing of the transgenic T cells may occur by any suitable method, but in particular embodiments, the gene editing of the transgenic T cells occurs by CRISPR methods. Thus, in particular embodiments the transgenic cells are exposed to suitable amounts of Cas9 and guide RNA. In certain cases the transgenic cells are exposed to suitable amounts of CpF1 and guide RNA. In cases wherein more than one gene of the T cells is to be disrupted for expression, there may be a group of guide RNAs that includes one or more sequence-specific guide RNAs for each of the desired genes to be edited. In particular embodiments, the T cells, including in some cases transgenic T cells, are subjected to two different electroporation steps that may or may not be separated in time by 1, 2, 3, or more days. In such cases, a first electroporation step comprises targeting one or more genes and a second or subsequent electroporation step comprises targeting one or more genes that are different genes than in an earlier electroporation step. In some cases, there are successive electroporation steps beyond two electroporation steps, including 3, 4, 5, or more additional electroporation steps. In any case wherein multiple electroporation steps are utilized, the next electroporation may occur only after a duration of certain time, such as only after 1, 2, 3, 4, or more days since the prior electroporation step.

In certain embodiments, the T cells undergo the gene editing step first, followed by the transduction or transfection step to harbor one or more heterologous antigen receptor genes. In such cases, the step for transduction or transfection step to harbor one or more heterologous antigen receptor genes may occur within 1, 2, 3, or more days following the gene editing step.

Following gene editing and transformation/transduction of the T cells, the T cells may or may not be subjected to a second expansion step to increase the number of transgenic gene edited T cells. In specific embodiments, a second expansion step in the process may be substantially identical to the first expansion step in the process, although in alternative embodiments the second expansion step is different than the first expansion step, such as having different media, different exogenously added compounds, different duration of time for culture/expansion, different expansion flasks such as GREX or WAVE to name a few or a combination thereof, a combination thereof, and so forth. In particular embodiments, the media in which the expansion step occurs comprises one or more agents to facilitate expansion, such as one or more cytokines. In specific embodiments, the concentration of cytokine(s) in the media is in the range of 100-300 units/mL, including 100, 125, 150, 175, 200, 225, 250, 275, or 300 units/mL, or 1-500 nM. The expansion step may occur at a certain temperature, such as about 35° C.-38° C., including 35° C., 36° C., 37° C., or 38° C. The expansion step may occur at a certain level of oxygen, such as from 3%-7% CO₂; in specific embodiments the expansion step occurs at 3%, 4%, 5%, 6%, or 7% CO₂. The expansion step may last for a particular duration of time, such as a certain number of days. In specific embodiments, the expansion step lasts for 4, 5, 6, 7, or more days, but in specific cases the expansion step lasts from 4-10, 4-9, 4-8, 4-7, 4-6, 4-5, 5-10, 5-9, 5-8, 5-6, 5-7, 6-10, 6-9, 6-8, 6-7, 8-10, 8-9, 9-10, or longer. In particular embodiments, the media in the expansion step may or may not be changed during the expansion, but in specific embodiments on day 3 after initiation of the expansion step the media is changed. In specific cases the cells are centrifuged and resuspended in the same or a similar media than before, including with a media comprising or comprising at least 100, 125, 150, 175, 200, 225, 250, 275, 300, 325, 350, 375, 400, 425, 450, 475, or 500 units/mL of cytokine(s), or more. In at least some cases, the method is performed in a bioreactor, including a gas permeable bioreactor. In particular aspects, the gas permeable bioreactor is G-Rex®100M or G-Rex100®. The expansion may or may not occur in a particular volume, such as 3-5 L and any range derivable therein.

In certain embodiments, there is an in vitro method of producing engineered T cells, comprising the steps of: (a) optionally exposing T cells from a mixture of cells to negative or positive selection to enrich the T cells; (b) optionally expanding for a first time period T cells with an effective amount of one or more of interleukin (IL)-2, IL-4, IL-7, IL-12, IL-15, IL-21 and/or one or more other T-cell tropic cytokines (s) to produce expanded T cells; and (c) delivering to the expanded T cells an effective amount of Cas9 or CpF1, and one or more guide RNAs to disrupt expression of one or more endogenous genes in the T cells, thereby producing gene edited T cells. In some embodiments, the method further comprises the step of (d) after a second time period, delivering to the gene edited T cells an effective amount of Cas9 or CpF1, and one or more guide RNAs to disrupt expression of one or more endogenous genes in the T cells, wherein the one or more guide RNAs in step (c) disrupt expression of a different gene or different genes than the one or more guide RNAs in step (d). In some cases, the first time period is 30-36 hours and/or the second time period is 2-3 days. Any method may further comprise the step of modifying the expanded T cells and/or the gene edited T cells to express one or more heterologous antigen receptors and/or one or more heterologous cytokines. In certain aspects of any method, at any time during the method the cells are expanded in the presence of one or more viral antigens and one or more cytokines to produce viral-specific cells. In specific cases, after any modifying step the cells are expanded in the presence of one or more viral antigens and one or more cytokines to produce viral-specific cells.

In particular embodiments, there is an in vitro or ex vivo method of producing engineered T cells, in no particular order comprising the steps of:

(a) optionally exposing T cells from a mixture of cells to negative or positive selection to enrich the T cells;

(b) expanding T cells with an effective amount of one or more of IL-2, IL-4, IL-7, IL-12, IL-15, IL-21 and/or one or more other T-cell tropic cytokines;

(c) delivering to T cells an effective amount of Cas9 or CpF1, and one or more guide RNAs to disrupt expression of one or more endogenous genes in the T cells;

(d) optionally delivering to the T cells an effective amount of Cas9 or CpF1, and one or more guide RNAs to disrupt expression of one or more endogenous genes in the T cells that are different from the one or more genes in a previous step;

(e) modifying the T cells to express one or more heterologous antigen receptors and/or one or more heterologous cytokines; and

(f) expanding the T cells in the presence of one or more viral antigens and optionally one or more cytokines to produce viral-specific cells.

In certain embodiments, 1, 2, 3, 4, 5, or all of steps (a)-(f) are utilized in the method and may be in any order. In some cases, steps (a), (b), (c), (d), (e), and (f) are utilized in any order. In some cases, steps (b), (c), (d), (e), and (f) are utilized in any order. In some cases, steps (a), (b), (c), (d), and (e) are utilized in any order. In some cases, steps (b), (c), (d), and (e) are utilized in any order. In some cases, steps (a), (c), (d), (e), and (f) are utilized in any order. In some cases, steps (a), (b), (c), (e), and (f) are utilized in any order. In some cases, steps (a), (b), (c), and (e) are utilized in any order. In some cases, steps (a), (b), and (c) are utilized in any order. In some cases, steps (a), (b), (c) and (d) are utilized in any order. In some cases, optionally (a), optionally (b), (c), optionally (d), (e), and (f) are utilized in any order. In some cases, optionally (a), optionally (b), (c), optionally (d), and one or both of (e) and (f) are utilized in any order.

In particular embodiments, the duration of each of the steps in the method may occur for 1-24 hours, 1-7 days, 1-4 weeks, and so forth, and any range derivable therein. In particular embodiments, the time period for a particular step is 1-3 days, 1-2 days, 2-3 days, or longer. In some embodiments, the time period for a particular step is 24-48 hours, 24-36 hours, 24-30 hours, 30-48 hours, 20-36 hours, and so forth.

Following production of the desired T cells, the cells may be utilized, analyzed, stored, a combination thereof, and so forth. In specific embodiments, the cells are analyzed for functionality, cytotoxicity, in vivo activity, a combination thereof, and so forth. For example, the cells may be analyzed for (1) the ability of the heterologous antigen receptor to bind its target antigen; (2) expression, or lack thereof, of the edited gene to confirm knockdown or knockout of expression; (3) anti-cancer activity; or (4) a combination thereof. In specific cases, the cells are subjected to mass spectrometry and/or RNA sequencing, for example.

In particular embodiments, the produced T cells are stored, including cryopreserved, for example. For example, the T cells may be cryopreserved for use by the individual from which the starting T cells were obtained, or the T cells may be cryopreserved for use by an individual that is different from which the starting T cells were obtained. The cells at any point during the process, including following the process, may be stored, such as cryopreserved, and then at the appropriate time of need delivered to an individual that donated the starting T cells, as in an autologous manner, or delivered to another individual from the donor that donated the starting T cells. As such. the cells may be used in an off-the-shelf manner and in some embodiments can be customized to the needs of a recipient individual, and this customization may occur prior to storage and/or following storage. The customization for an individual may include tailoring the cells to the therapeutic needs of an individual, such as engineering the cells to be specifically effective against a cancer of the individual or engineering the cells to be specifically effective against a viral infection of the individual. The same batch of produced T cells may be utilized for different individuals.

### III. Specific Embodiments

A specific embodiment for production of engineered T cells is as follows, although the following protocol may be altered or optimized in a variety of routine ways:

T cell expansion (includes all T cells, viral-specific T cells, tumor-infiltrating lymphocytes (TILs), and CAR T cells or TCR T cells)+CRISPR gene editing

CRISPR Gene Editing of T Cells

One procedure for generating primary human T-cells from any source (peripheral blood, cord blood, tumor infiltrating lymphocytes, bone marrow or T-cell lines) with CRISPR Cas9 editing is as follows:

On Day 0, T cells from any source are negatively selected using the GMP T cell isolation kit from Miltenyi. Then cells are resuspended in RPMI plus human serum AB (10%) at 0.5-1 million/mL. Next, stimulate T cells with anti-CD3/CD28 beads in the presence of IL-2 (50 iU/mL) for 30-36 hrs, after which the cells are ready for CRISPR gene editing. If editing one or two genes, perform electroporation for CRISPR-Cas9 at 30-36 hrs. If targeting more than two genes, rest cells after the first electroporation for 2-3 days and then perform second CRISPR+electroporation with desired gRNA. (see below for details Crispr Cas9 application)

Viral-Specific T Cells+CRISPR Gene Editing

The procedures for generating virus-specific T cells with CRISPR Cas9 gene editing is as follows:

On Day 0, mononuclear cells are isolated from healthy donors. Split PBMC into different aliquots and stimulate separately with individual virus specific pepmix (GMP grade) derived from CMV, adenovirus, BK virus, EBV, HHV6, influenza, SARS-CoV-2 (COVID-19), JC virus, adenovirus, EBV, etc. (1 ug/ml, JPT) in one milliliter of Click/RPMI media for two hour in 37° C. After 2 hours incubation, bring the volume to have the cells at 0.5×10⁶/ml in media and add cytokines:

For cells stimulated with CMV pepmix add IL4 60 ng/ml plus IL7 10 ng/ml,

For cells stimulated with BKV pepmix or JCV pepmix add IL2 50 IU/ml and IL7 10 ng/ml and IL15 10 ng/ml,

For cells stimulated with adenovirus pepmix add IL4 60 ng/ml plus IL7 10 ng/ml,

For cells stimulated with EBV pepmix add IL4 60 ng/ml plus IL7 10 ng/ml,

In specific embodiments, one makes a multivirus specific T-cell line by combining any of the pepmixes for the viruses mentioned above at (1 ug/ml) with combinations of cytokines above.

Change media every other day in the presence of cytokines. If editing one or two genes, on day 7 of culture (can do up to day 10) perform electroporation for CRISPR-Cas9. If targeting more than two genes, rest cells after the first electroporation step on day 7 and perform a second CRISPR+electroporation with desired gRNA on Days 10-11. (see below for details CRISPR Cas9 application).

CAR T Cells+CRISPR Gene Editing

The procedure for generating the genetically modified CAR T cells from any source (peripheral blood, cord blood, tumor infiltrating lymphocytes, bone marrow) with CRISPR Cas9 editing is as follows:

On Day 0, mononuclear cells are isolated from a donor and negatively selected using the GMP T cell isolation kit from Miltenyi. Then cells are resuspended in RPMI plus human serum AB (10%) at 0.5-1 million/mL. Next stimulate T cells with anti-CD3/CD28 beads in the presence of IL-2 (50 iU/mL) for 30-36 hrs, after which the cells are ready for CAR transduction or CRISPR gene editing (in some embodiments CRISRP gene editing is performed first, followed by CAR transduction on days 4-5; in some embodiments CAR transduction is performed first followed by CRISPR gene editing on day 4-5).

For CAR transduction, a retronectin transduction plate is prepared by incubating a non-tissue culture plate containing 1 ml of 1% retronectin diluted in PBS per well for 5 hours at 37° C. The retronectin plate is then aspirated at room temperature and complete media is added to the wells. The plate containing media is incubated for 10 minutes. The media is then replaced with the retroviral supernatant and centrifuged at 2000 g at 32° C. for two hours. Next, the retroviral supernatant is replaced with fresh retroviral supernatant and the T cell suspension containing 0.5×10⁶ cells is added to each well, along with a suitable cytokine condition. The plate is centrifuged at 2000 g at 32° C. for 30 minutes, then incubated at 37° C./5% CO₂. Two days after transduction, one can check transduction efficiency and perform electroporation for CRISPR-Cas9 gene editing. If editing one or two genes, on day 4-5 of culture one can perform electroporation for CRISPR-Cas9 gene editing. If targeting more than two genes, one can rest cells for 2-3 days after the first electroporation and then perform a second CRISPR-Cas9 gene editing step with the desired gRNA. (see below for details CRISPR-Cas9 application)

Virus-Specific CAR T Cells+CRISPR-Cas9 Gene Editing

The procedures for generating virus-specific T cells that are also genetically modified to express a CAR (to allow targeting of both viruses using endogenous TCR and antigen targets using CAR) as well as CRISPR-Cas9 gene editing is as follows:

Follow the procedure for viral-specific T cell generation as described above. Perform CAR retroviral transduction on day 7-8 of VST culture. First prepare a retronectin transduction plate by incubating a non-tissue culture plate containing 1 ml of 1% retronectin diluted in PBS per well for 5 hours in 37° C. The retronectin plate is then aspirated and complete media is added to the wells. The plate containing media is incubated for 10 minutes. The media is then replaced with the retroviral supernatant and centrifuged at 2000 g at 32° C. for two hours. Next, the retroviral supernatant is replaced with fresh retroviral supernatant and the T cell suspension containing 0.5×10⁶ cells is added to each well, along with suitable cytokine condition. The plate is centrifuged at 2000 g at 32° C. for 30 minutes, then incubated at 37° C. with 5% CO₂. Two days after transduction one can check the transduction efficiency and perform electroporation for CRISPR-CAS9 gene editing.

In some embodiments, CRISRP-Cas9 gene editing step is performed first (on days 7-8) followed by CAR transduction 2-3 days later.

If editing one or two genes, on day 9-10 of culture (can do up to day 14) perform electroporation for CRISP-Cas9 if desired. If targeting more than two genes, rest cells after the first electroporation step for 2-3 days and then perform second CRISPR Cas9 gene editing step+electroporation with the desired gRNA. (see below for details CRISPR Cas9 application). (see below for details CRISPR-Cas9 application)

1. crRNA Pre-Complexing and Electroporation (Lonza 4D) (for 5-100 Million T Cells)

Step 1: Make crRNA+tracrRNA duplex

The starting concentration of crRNA and tracrRNA are 200 uM. The final
concentration after mixing them in equimolar concentration is 100 uM.

| | | concen- | | | concen- |
|---|---|---|---|---|---|
| | volume | tration | | volume | tration |
| crRNA # 1 | 5 | 200 uM | crRNA # 2 | 5 | 200 uM |
| tracrRNA | 5 | 200 uM | tracrRNA | 5 | 200 uM |
| IDTE Buffer | 0 | | IDTE Buffer | 0 | |
| total volume | 10 ul | 100 uM | total volume | 10 ul | 100 uM |

a. Mix with pipette, and centrifuge.
b. Incubate at 95° C. for 5 min in thermocycler.
c. Allow to cool to room temperature on the benchtop

Step 2: Combine the crRNA: tracrRNA duplex and Cas9 Nuclease

| | volume | | volume | |
|---|---|---|---|---|
| crRNA # 1: tracrRNA | 1.2 | ul | 2.4 | ul |
| duplex (Step 1) | (120 | pmol) | | |
| Cas9 (Undiluted) | 1.7 | ul | 3.4 | ul |
| | (104 | pmol) | | |
| P3 Buffer | 2.1 | | 4.2 | |
| total volume | 5 | ul | 10 | ul |

| | volume | | volume | |
|---|---|---|---|---|
| crRNA # 2: tracrRNA | 1.2 | ul | 2.4 | ul |
| duplex (Step 1) | (120 | pmol) | | |
| Cas9 (Step 2) | 1.7 | ul | 3.4 | ul |
| | (104 | pmol) | | |
| P3 Buffer | 2.1 | | 4.2 | |
| total volume | 5 | ul | 10 | ul |

a. Mix with pipette, and centrifuge.
b. Incubate the mixture at room temperature for 15 min

Step 3: Combine crRNA #1 and crRNA #2 from Step 3

| | volume | volume |
|---|---|---|
| crRNA # 1 + tracrRNA + cas9 (Step 3) | 5 ul | 10 ul |
| crRNA # 2 + tracrRNA + cas9 (Step 3) | 5 ul | 10 ul |
| total volume | 10 ul | 20 ul |

| | Volume |
|---|---|
| crRNA #1, 2 + tracrRNA + cas9 (Step 3) | 20 ul |
| Cell Suspension | 100 ul |
| Total volume | 120 ul |

Step 4: Perform electroporation
a. Prepare culture plate with media (preferentially antibiotic free), with IL-2
b. Prepare 5E+106 cells (wash twice with PBS to remove FBS) and re-suspend in 100 ul P3 primary cell Nucleofector Solution just before use.
c. Mix Cell suspension and RNP (final Cas9 concentration is 4.6 uM, gRNA concentration is 4 uM), transfer to nucluocuvette and click lid into place.
d. The electroporation program is EO-115 the cells are then added to the culture plate and allowed to recover in 37 C° incubator.
e. Up to 5×10⁶-X unit (Cat: AAF-1002X)
f. To electroporate up to 30×10⁶, use the 1 ml LV Kit L Unit (Cat. #: V4LC-2002).
g. To electroporate up to 100×10⁶ or more, use the 1 ml LV Kit L Unit (Cat. #: AAF-1002L).

CRISPR CAS9: Small Scale Protocol (Starting Cell Population 0.25-3 Million)

1. sgRNA-Cas9 Pre-Complexing and Electroporation (Neon-Thermo Fisher)
a. 1 or 2 sgRNAs are designed and used for each gene and incubated on ice for 20 minutes.
b. After 20 minutes, the sgRNA are added to 250,000 T-cells re-suspended in T-buffer* (included with Neon Electroporation Kit, Invitrogen, total volume including RNP complex and cells should be 14 ul) and electroporated with 10 ul electroporation tip using the Neon Transfection System.
c. The electroporation conditions are 1600V, 10 ms, and 3 pulses. The cells are then added to the culture plate with media and 50 IU/ml IL2 and rested in an incubator at 37° C.
   2. crRNA Pre-Complexing and Electroporation (Neon-Thermo Fisher)

Step 1: Make crRNA+tracrRNA duplex

The starting concentration of crRNA and tracrRNA are 200 uM. The final
concentration after mixing them in equimolar concentration is 44 uM.

| | | concen- | | | concen- |
|---|---|---|---|---|---|
| | volume | tration | | volume | tration |
| crRNA # 1 | 2.2 ul | 200 uM | crRNA # 2 | 2.2 ul | 200 uM |
| tracrRNA | 2.2 ul | 200 uM | tracrRNA | 2.2 ul | 200 uM |
| IDTE Buffer | 5.6 ul | | IDTE Buffer | 5.6 ul | |
| total volume | 10 ul | 44 uM | total volume | 10 ul | 44 uM |

a. Mix with pipette, and centrifuge.
b. Incubate at 95° C. for 5 min in a thermocycler.
c. Allow to cool to room temperature on the benchtop.

Step 2: Prepare cas9 nuclease

| | volume | |
|---|---|---|
| Alt-R S.p. Cas9 Nuclease 3NLS (61 uM) | 3 | ul |
| T buffer | 7 | ul |
| total volume | 10 | ul |
| final concentration | 18 | uM |

Step 3: Combine the crRNA: tracrRNA duplex and Cas9 Nuclease

| | volume |
|---|---|
| crRNA # 1: tracrRNA duplex | 2 ul |
| (Step 1) | |
| Cas9 (Step 2) | 2 ul |
| total volume | 4 ul |

| | volume |
|---|---|
| crRNA # 2: tracrRNA duplex | 2 ul |
| (Step 1) | |
| Cas9 (Step 2) | 2 ul |
| total volume | 4 ul |

a. Mix with pipette, and centrifuge.
b. Incubate the mixture at room temperature for 15 min

Step 4: Combine crRNA #1 and crRNA #2 from Step 3

| | volume |
|---|---|
| crRNA # 1 + tracrRNA + | 2.25 ul |
| cas9 (Step 3) | |
| crRNA # 2 + tracrRNA + | 2.25 ul |
| cas9 (Step 3) | |
| total volume | 4.5 ul |

Step 5: Perform electroporation
Prepare 250,000 cells per well and re-suspend in 7.5 ul of T buffer just before use.
The electroporation conditions are 1600V, 10 ms, and 3 pulses. The cells are then added to the culture plate and allowed to rest in an incubator at 37° C.

### IV. T Cells

Embodiments of the disclosure utilize T cells, including their expansion and modification. The T cells may be viral-specific T cells, CAR-specific T cells, TCR T cells, tumor-infiltrating lymphocytes, regulatory T cells, CD8+ T cells, CD4+ T cells, gamma delta T cells, or a mixture thereof, and so on. The T cells may be expanded by particular methods disclosed herein, including methods tailored to the type of T cells to be produced.

Some embodiments encompassed herein utilize one or more of the several basic approaches for the derivation, activation and expansion of functional anti-tumor effector cells that have been described in the last two decades. These approaches include: autologous cells, such as tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. These approaches have given rise to numerous protocols for T cell preparation and immunization that can be used in the methods described herein.

In some embodiments, the T cells are derived from peripheral blood, cord blood, tumor-infiltrating lymphocytes, bone marrow, lymph, umbilical cord, lymphoid organs, induced pluripotent stem cells, hematopoietic stem cells, or a mixture thereof. In some aspects, the cells are human cells. The cells may be primary cells, such as those isolated directly from a subject and/or isolated from a subject and frozen. In some embodiments, the cells include one or more subsets of T cells or other cell types, such as whole T cell populations, CD4⁺ cells, CD8⁺ cells, regulatory T cells, gamma delta T cells, and subpopulations thereof, such as those defined by function, activation state, maturity, potential for differentiation, expansion, recirculation, localization, and/or persistence capacities, antigen specificity, type of antigen receptor, presence in a particular organ or compartment, marker or cytokine secretion profile, and/or degree of differentiation. With reference to the subject to be treated, the cells may be allogeneic, autologous, or a mixture thereof. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells (e.g., CD4⁺ and/or CD8⁺ T cells) are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, one or more of the T cell populations is enriched for or depleted of cells that are positive for one or more specific markers, such as surface markers, or that are negative for one or more specific markers. In some cases, such markers are those that are absent or expressed at relatively low levels on certain populations of T cells (e.g., non-memory cells) but are present or expressed at relatively higher levels on certain other populations of T cells (e.g., memory cells).

In some embodiments, T cells are separated from a PBMC sample by negative selection of one or more markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for one or more markers expressed or expressed to a relatively higher degree on one or more naive, memory, and/or effector T cell subpopulations.

In some embodiments, CD8⁺ T cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations.

In some embodiments, the T cells are autologous T cells. Such methods may be used, for example, for treating an individual having cancer. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests are cultured in interleukin-2 (IL-2).

Any cultured T cells can be pooled and rapidly expanded. Rapid expansion provides an increase in the number of antigen-specific T-cells of at least about 50-fold (e.g., 50-, 60-, 70-, 80-, 90-, or 100-fold, or greater) over a period of about 10 to about 14 days. In particular embodiments, rapid expansion provides an increase of at least about 200-fold (e.g., 200-, 300-, 400-, 500-, 600-, 700-, 800-, 900-, or greater) over a period of about 10 to about 14 days.

Expansion can be accomplished by any of a number of methods as are known in the art. For example, T cells can be rapidly expanded using non-specific T-cell receptor stimulation in the presence of feeder lymphocytes and either (as examples only) interleukin-2 (IL-2) or interleukin-15 (IL-15). The non-specific T-cell receptor stimulus can include around 30 ng/ml of OKT3, a mouse monoclonal anti-CD3 antibody (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as an human leukocyte antigen A2 (HLA-A2) binding peptide, in the presence of a T-cell growth factor, such as 300 IU/ml IL-2 or IL-15, with IL-2 being preferred. The in vitro-induced T-cells are rapidly expanded by re-stimulation with the same antigen(s) of the cancer pulsed onto HLA-A2-expressing antigen-presenting cells. Alternatively, the T-cells can be re-stimulated with irradiated, autologous lymphocytes or with irradiated HLA-A2⁺ allogeneic lymphocytes and IL-2, for example.

The T cells can be modified to express a T cell growth factor that promotes the growth and activation of the autologous T cells. Suitable T cell growth factors include, for example, interleukin (IL)-2, IL-7, IL-15, IL-12, and IL-21. Suitable methods of modification are known in the art. See, for instance, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, N Y, 1994. In particular aspects, modified autologous T cells express the T cell growth factor at high levels. T cell growth factor coding sequences, such as that of IL-12, are readily available in the art, as are promoters, the operable linkage of which to a T cell growth factor coding sequence promote high-level expression.

### V. Heterologous Antigen Receptors

The T cells of the present disclosure can be genetically engineered to express one or more heterologous antigen receptors, such as engineered TCRs, CARs, chimeric cytokine receptors, chemokine receptors, a combination thereof, and so on. The heterologous antigen receptors are synthetically generated by the hand of man. In particular embodiments, the T cells are modified to express one or more CAR and/or TCR having antigenic specificity for a cancer antigen. Multiple CARs and/or TCRs, such as to different antigens, may be added to the T cells. In some aspects, the immune cells are engineered to express the CAR or TCR by knock-in of the CAR or TCR at a particular gene locus, such as by using CRISPR.

Although the T cells are particularly edited using CRISPR, alternative suitable methods of modification are known in the art. See, for instance, Sambrook and Ausubel, supra. For example, the cells may be transduced to express a TCR having antigenic specificity for a cancer antigen using transduction techniques described in Heemskerk et al., 2008 and Johnson et al., 2009. In some embodiments, the cells comprise one or more nucleic acids introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

In some embodiments, the CAR comprises one or more extracellular antigen-recognition domains that specifically bind to one or more corresponding antigens. In some embodiments, the antigen is a protein expressed on the surface of cells, such as the surface of cancer cells. In some embodiments, although one example of a CAR comprises: a) one or more intracellular signaling domains, b) a transmembrane domain, and c) an extracellular domain comprising one or more antigen binding regions, in other cases the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary antigen receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., 2013; Davila et al., 2013; Turtle et al., 2012; Wu et al., 2012. In some aspects, the genetically engineered antigen receptors include a CAR as described in U.S. Pat. No. 7,446,190, and those described in International Patent Application Publication No.: WO/2014055668 A1.

A. Chimeric Antigen Receptors (CARs)

In some embodiments, the T cells are engineered to express one or more CARs. One example of a CAR comprises: a) one or more intracellular signaling domains, b) a transmembrane domain, and c) an extracellular domain comprising one or more antigen binding regions.

In some embodiments, the engineered antigen receptors include CARs, including activating or stimulatory CARs, costimulatory CARs (see WO2014/055668), and/or inhibitory CARs (iCARs, see Fedorov et al., 2013). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

Certain embodiments of the present disclosure concern the use of nucleic acids, including nucleic acids encoding an antigen-specific CAR polypeptide, including a CAR that has been humanized to reduce immunogenicity (hCAR), comprising an intracellular signaling domain, a transmembrane domain, and an extracellular domain comprising one or more signaling motifs. In certain embodiments, the CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human CAR nucleic acids may be human genes used to enhance cellular immunotherapy for human patients. In a specific embodiment, the invention includes a full-length CAR cDNA or coding region. The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha.

In some embodiments, the CAR nucleic acid comprises a sequence encoding other costimulatory receptors, such as a transmembrane domain and a modified CD28 intracellular signaling domain. Other costimulatory receptors include, but are not limited to one or more of CD28, CD27, OX-40 (CD134), DAP10, DAP12, and 4-1BB (CD137). In addition to a primary signal initiated by CD3ζ, an additional signal provided by a human costimulatory receptor inserted in a human CAR is important for full activation of T cells and could help improve in vivo persistence and the therapeutic success of the adoptive immunotherapy.

In some embodiments, CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In certain embodiments of the chimeric antigen receptor, the antigen-specific portion of the receptor (which may be referred to as an extracellular domain comprising an antigen binding region) comprises a tumor associated antigen or a pathogen-specific antigen binding domain. Antigens include carbohydrate antigens recognized by pattern-recognition receptors, such as Dectin-1. A tumor associated antigen may be of any kind so long as it is expressed on the cell surface of tumor cells. Exemplary embodiments of tumor associated antigens include CD19, CD20, carcinoembryonic antigen, alphafetoprotein, CA-125, MUC-1, CD56, EGFR, c-Met, AKT, Her2, Her3, epithelial tumor antigen, melanoma-associated antigen, mutated p53, mutated ras, and so forth. In certain embodiments, the CAR may be co-expressed with a cytokine to improve persistence when there is a low amount of tumor-associated antigen. For example, CAR may be co-expressed with one or more cytokines, such as IL-7, IL-2, IL-15, IL-12, IL-18, IL-21, or a combination thereof.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, and DAP molecules. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

In certain embodiments, the platform technologies disclosed herein to genetically modify immune cells, such as T cells, comprise (i) non-viral gene transfer using an electroporation device (e.g., a nucleofector), (ii) CARs that signal through endodomains (e.g., CD28/CD3-, CD137/CD3-, or other combinations), (iii) CARs with variable lengths of extracellular domains connecting the antigen-recognition domain to the cell surface, and, in some cases, (iv) artificial antigen presenting cells (aAPC) derived from K562 to be able to robustly and numerically expand CARP immune cells (Singh et al., 2008; Singh et al., 2011).

B. T Cell Receptor (TCR)

In particular embodiments, engineered T cells are produced using the disclosed methods wherein the T cells express a non-natural TCR, compared to their natural, endogenous TCR. In some embodiments, the genetically engineered antigen receptors include recombinant TCRs and/or TCRs cloned from naturally occurring T cells. A “T cell receptor” or “TCR” refers to a molecule that contains a variable a and β chains (also known as TCRα and TCRβ, respectively) or a variable γ and δ chains (also known as TCRγ and TCRδ, respectively) and that is capable of specifically binding to an antigen peptide bound to a MHC receptor. In some embodiments, the TCR is in the αβ form.

Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., a-chain, β-chain) can contain two immunoglobulin domains, a variable domain (e.g., Vₐ or Vp; typically amino acids 1 to 116 based on Kabat numbering Kabat et al., “Sequences of Proteins of Immunological Interest, US Dept. Health and Human Services, Public Health Service National Institutes of Health, 1991, 5th ed.) at the N-terminus, and one constant domain (e.g., a-chain constant domain or Cₐ, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains a and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005). In some embodiments, phage display is used to isolate TCRs against a target antigen (see, e.g., Varela-Rohena et al., 2008 and Li et al., 2005). In some embodiments, the TCR or antigen-binding portion thereof can be synthetically generated from knowledge of the sequence of the TCR.

C. Antigens

Among the antigens targeted by the genetically engineered heterologous antigen receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In some embodiments, the diseases and conditions are infectious diseases, such as viral infections. In other embodiments, the antigen is expressed on normal cells and/or is expressed on the engineered cells.

Any suitable antigen may be targeted in the present method. The antigen may be associated with certain cancer cells but not associated with non-cancerous cells, in some cases. Exemplary antigens include, but are not limited to, antigenic molecules from infectious agents, auto-/self-antigens, tumor-/cancer-associated antigens, and tumor neoantigens (Linnemann et al., 2015). In particular aspects, the antigens include NY-ESO, EGFRvIII, Muc-1, Her2, CA-125, WT-1, Mage-A3, Mage-A4, Mage-A10, TRAIL/DR4, and CEA. In particular aspects, the antigens for the two or more antigen receptors include, but are not limited to, CD19, EBNA, WT1, CD123, NY-ESO, EGFRvIII, MUC1, HER2, CA-125, WT1, Mage-A3, Mage-A4, Mage-A10, TRAIL/DR4, and/or CEA. The sequences for these antigens are known in the art, for example, in the GenBank® database: CD19 (Accession No. NG_007275.1), EBNA (Accession No. NG_002392.2), WT1 (Accession No. NG_009272.1), CD123 (Accession No. NC_000023.11), NY-ESO (Accession No. NC_000023.11), EGFRvIII (Accession No. NG_007726.3), MUC1 (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Tumor-associated antigens may be derived from prostate, breast, colorectal, lung, pancreatic, renal, mesothelioma, ovarian, liver, brain, bone, stomach, spleen, testicular, cervical, anal, gall bladder, thyroid, or melanoma cancers, as examples. Exemplary tumor-associated antigens or tumor cell-derived antigens include MAGE 1, 3, and MAGE 4 (or other MAGE antigens such as those disclosed in International Patent Publication No. WO 99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self-peptide hormone, such as whole length gonadotrophin hormone releasing hormone (GnRH), a short 10 amino acid long peptide, useful in the treatment of many cancers.

Tumor antigens include tumor antigens derived from cancers that are characterized by tumor-associated antigen expression, such as HER-2/neu expression. Tumor-associated antigens of interest include lineage-specific tumor antigens such as the melanocyte-melanoma lineage antigens MART-1/Melan-A, gp100, gp75, mda-7, tyrosinase and tyrosinase-related protein.

Illustrative tumor-associated antigens include, but are not limited to, tumor antigens derived from or comprising any one or more of, CD19, EBNA, CD123, HER2, CA-125, TRAIL/DR4, CD20, CD70, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, -2, -8, GAGE-3, -4, -5, -6, -7B, NA88-A, MC1R, mda-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), VEGFR2, cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), NY ESO 1, c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SAGE, SART3, STn, PAX3, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, and LRRN1.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

In other embodiments, an antigen is obtained or derived from a pathogenic microorganism or from an opportunistic pathogenic microorganism (also called herein an infectious disease microorganism), such as a virus, fungus, parasite, and bacterium. In certain embodiments, antigens derived from such a microorganism include full-length proteins.

Illustrative pathogenic organisms whose antigens are contemplated for use in the method described herein include human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), polyomavirus (e.g., BK virus and JC virus), adenovirus, Staphylococcus species including Methicillin-resistant Staphylococcus aureus (MRSA), and Streptococcus species including Streptococcus pneumoniae. As would be understood by the skilled person, proteins derived from these and other pathogenic microorganisms for use as antigen as described herein and nucleotide sequences encoding the proteins may be identified in publications and in public databases such as GENBANK®, SWISS-PROT®, and TREMBL®.

Antigens derived from human immunodeficiency virus (HIV) include any of the HIV virion structural proteins (e.g., gp120, gp41, p17, p24), protease, reverse transcriptase, or HIV proteins encoded by tat, rev, nef, vif, vpr and vpu.

Antigens derived from herpes simplex virus (e.g., HSV 1 and HSV2) include, but are not limited to, proteins expressed from HSV late genes. The late group of genes predominantly encodes proteins that form the virion particle. Such proteins include the five proteins from (UL) which form the viral capsid: UL6, UL18, UL35, UL38 and the major capsid protein UL19, UL45, and UL27, each of which may be used as an antigen as described herein. Other illustrative HSV proteins contemplated for use as antigens herein include the ICP27 (H1, H2), glycoprotein B (gB) and glycoprotein D (gD) proteins. The HSV genome comprises at least 74 genes, each encoding a protein that could potentially be used as an antigen.

Antigens derived from cytomegalovirus (CMV) include CMV structural proteins, viral antigens expressed during the immediate early and early phases of virus replication, glycoproteins I and III, capsid protein, coat protein, lower matrix protein pp65 (ppUL83), p52 (ppUL44), IE1 and 1E2 (UL123 and UL122), protein products from the cluster of genes from UL128-UL150 (Rykman, et al., 2006), envelope glycoprotein B (gB), gH, gN, and pp150. As would be understood by the skilled person, CMV proteins for use as antigens described herein may be identified in public databases such as GENBANK®, SWISS-PROT®, and TREMBL® (see e.g., Bennekov et al., 2004; Loewendorf et al., 2010; Marschall et al., 2009).

Antigens derived from Epstein-Ban virus (EBV) that are contemplated for use in certain embodiments include EBV lytic proteins gp350 and gp110, EBV proteins produced during latent cycle infection including Epstein-Ban nuclear antigen (EBNA)-1, EBNA-2, EBNA-3A, EBNA-3B, EBNA-3C, EBNA-leader protein (EBNA-LP) and latent membrane proteins (LMP)-1, LMP-2A and LMP-2B (see, e.g., Lockey et al., 2008).

Antigens derived from respiratory syncytial virus (RSV) that are contemplated for use herein include any of the eleven proteins encoded by the RSV genome, or antigenic fragments thereof: NS 1, NS2, N (nucleocapsid protein), M (Matrix protein) SH, G and F (viral coat proteins), M2 (second matrix protein), M2-1 (elongation factor), M2-2 (transcription regulation), RNA polymerase, and phosphoprotein P.

Antigens derived from Vesicular stomatitis virus (VSV) that are contemplated for use include any one of the five major proteins encoded by the VSV genome, and antigenic fragments thereof: large protein (L), glycoprotein (G), nucleoprotein (N), phosphoprotein (P), and matrix protein (M) (see, e.g., Rieder et al., 1999).

Antigens derived from an influenza virus that are contemplated for use in certain embodiments include hemagglutinin (HA), neuraminidase (NA), nucleoprotein (NP), matrix proteins M1 and M2, NS1, NS2 (NEP), PA, PB1, PB1-F2, and PB2.

Antigens derived from COVID-19 include the structural proteins spike (S), membrane (M), envelope (E) and nucleocapsid (N), as well as non-structural proteins.

Exemplary viral antigens also include, but are not limited to, adenovirus polypeptides, alphavirus polypeptides, calicivirus polypeptides (e.g., a calicivirus capsid antigen), coronavirus polypeptides, distemper virus polypeptides, Ebola virus polypeptides, enterovirus polypeptides, flavivirus polypeptides, hepatitis virus (AE) polypeptides (a hepatitis B core or surface antigen, a hepatitis C virus E1 or E2 glycoproteins, core, or non-structural proteins), herpesvirus polypeptides (including a herpes simplex virus or varicella zoster virus glycoprotein), infectious peritonitis virus polypeptides, leukemia virus polypeptides, Marburg virus polypeptides, orthomyxovirus polypeptides, papilloma virus polypeptides, parainfluenza virus polypeptides (e.g., the hemagglutinin and neuraminidase polypeptides), paramyxovirus polypeptides, parvovirus polypeptides, pestivirus polypeptides, picorna virus polypeptides (e.g., a poliovirus capsid polypeptide), pox virus polypeptides (e.g., a vaccinia virus polypeptide), rabies virus polypeptides (e.g., a rabies virus glycoprotein G), reovirus polypeptides, retrovirus polypeptides, and rotavirus polypeptides.

In certain embodiments, the antigen may be a bacterial antigen. In certain embodiments, a bacterial antigen of interest may be a secreted polypeptide. In other certain embodiments, bacterial antigens include antigens that have a portion or portions of the polypeptide exposed on the outer cell surface of the bacteria.

Antigens derived from Staphylococcus species including Methicillin-resistant Staphylococcus aureus (MRSA) that are contemplated for use include virulence regulators, such as the Agr system, Sar and Sae, the Arl system, Sar homologues (Rot, MgrA, SarS, SarR, SarT, SarU, SarV, SarX, SarZ and TcaR), the Srr system and TRAP. Other Staphylococcus proteins that may serve as antigens include Clp proteins, HtrA, MsrR, aconitase, CcpA, SvrA, Msa, CfvA and CfvB (see, e.g., Staphylococcus: Molecular Genetics, 2008 Caister Academic Press, Ed. Jodi Lindsay). The genomes for two species of Staphylococcus aureus (N315 and Mu50) have been sequenced and are publicly available, for example at PATRIC (PATRIC: The VBI PathoSystems Resource Integration Center, Snyder et al., 2007). As would be understood by the skilled person, Staphylococcus proteins for use as antigens may also be identified in other public databases such as GenBank®, Swiss-Prot®, and TrEMBL®.

Antigens derived from Streptococcus pneumoniae that are contemplated for use in certain embodiments described herein include pneumolysin, PspA, choline-binding protein A (CbpA), NanA, NanB, SpnHL, PavA, LytA, Pht, and pilin proteins (RrgA; RrgB; RrgC). Antigenic proteins of Streptococcus pneumoniae are also known in the art and may be used as an antigen in some embodiments (see, e.g., Zysk et al., 2000). The complete genome sequence of a virulent strain of Streptococcus pneumoniae has been sequenced and, as would be understood by the skilled person, S. pneumoniae proteins for use herein may also be identified in other public databases such as GENBANK®, SWISS-PROT®, and TREMBL®. Proteins of particular interest for antigens according to the present disclosure include virulence factors and proteins predicted to be exposed at the surface of the pneumococci (see, e.g., Frolet et al., 2010).

Examples of bacterial antigens that may be used as antigens include, but are not limited to, Actinomyces polypeptides, Bacillus polypeptides, Bacteroides polypeptides, Bordetella polypeptides, Bartonella polypeptides, Borrelia polypeptides (e.g., B. burgdorferi OspA), Brucella polypeptides, Campylobacter polypeptides, Capnocytophaga polypeptides, Chlamydia polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides (e.g., H. influenzae type b outer membrane protein), Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides (i.e., S. pneumoniae polypeptides), Proteus polypeptides, Pseudomonas polypeptides, Rickettsia polypeptides, Rochalimaea polypeptides, Salmonella polypeptides, Shigella polypeptides, Staphylococcus polypeptides, group A streptococcus polypeptides (e.g., S. pyogenes M proteins), group B streptococcus (S. agalactiae) polypeptides, Treponema polypeptides, and Yersinia polypeptides (e.g., Y pestis F1 and V antigens).

Examples of fungal antigens include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvularia polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite antigens include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides. Examples of helminth parasite antigens include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides. (e.g., P. falciparum circumsporozoite (PfCSP)), sporozoite surface protein 2 (PfSSP2), carboxyl terminus of liver state antigen 1 (PfLSA1 c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of ectoparasite antigens include, but are not limited to, polypeptides (including antigens as well as allergens) from fleas; ticks, including hard ticks and soft ticks; flies, such as midges, mosquitoes, sand flies, black flies, horse flies, horn flies, deer flies, tsetse flies, stable flies, myiasis-causing flies and biting gnats; ants; spiders, lice; mites; and true bugs, such as bed bugs and kissing bugs.

D. Suicide Genes

In some cases, any cells of the disclosure are modified to produce one or more agents other than heterologous cytokines, engineered receptors, and so forth. In specific embodiments, the cells, such as T cells, are engineered to harbor one or more suicide genes, and the term “suicide gene” as used herein is defined as a gene which, upon administration of a prodrug, effects transition of a gene product to a compound which kills its host cell. In some cases, the T cell therapy may be subject to utilization of one or more suicide genes of any kind when an individual receiving the T cell therapy and/or having received the T cell therapy shows one or more symptoms of one or more adverse events, such as cytokine release syndrome, neurotoxicity, anaphylaxis/allergy, and/or on-target/off tumor toxicities (as examples) or is considered at risk for having the one or more symptoms, including imminently. The use of the suicide gene may be part of a planned protocol for a therapy or may be used only upon a recognized need for its use. In some cases the cell therapy is terminated by use of agent(s) that targets the suicide gene or a gene product therefrom because the therapy is no longer required.

Examples of suicide genes include engineered nonsecretable (including membrane bound) tumor necrosis factor (TNF)-alpha mutant polypeptides (see PCT/US19/62009, which is incorporated by reference herein in its entirety), and they may be targeted by delivery of an antibody that binds the TNF-alpha mutant. Examples of suicide gene/prodrug combinations that may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be utilized. Other suicide genes include CD20, CD52, inducible caspase 9, purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP), as examples.

E. Methods of Delivery

In some embodiments, the T cells are modified to express one or more heterologous proteins, and these may be delivered to the T cells by any suitable method, including by one or more vectors. One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure. Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Ban virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

In specific embodiments, the vector is a multicistronic vector, such as is described in PCT/US19/62014, which is incorporated by reference herein in its entirety. In such cases, a single vector may encode the CAR or TCR (and the expression construct may be configured in a modular format to allow for interchanging parts of the CAR or TCR), a suicide gene, and one or more cytokines.

1. Viral Vectors

Viral vectors encoding an antigen receptor may be provided in certain aspects of the present disclosure. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor mediated-endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present invention are described below.

Lentiviruses are complex retroviruses, which, in addition to the common retroviral genes gag, pol, and env, contain other genes with regulatory or structural function. Lentiviral vectors are well known in the art (see, for example, U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

a. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters.

b. Promoter/Enhancers

The expression constructs provided herein comprise a promoter to drive expression of the antigen receptor. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30110 bp-upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the βlactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter, GADPH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at GenBank®, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

c. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Additionally, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the F2A (Foot-and-mouth disease virus 2A) or a “2A-like” sequence (e.g., Thosea asigna virus 2A; T2A).

d. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

e. Selection and Screenable Markers

In some embodiments, cells containing a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

2. Other Methods of Nucleic Acid Delivery

In addition to viral delivery of the nucleic acids encoding the antigen receptor, the following are additional methods of recombinant gene delivery to a given host cell and are thus considered in the present disclosure.

Introduction of a nucleic acid, such as DNA or RNA, into the immune cells of the current disclosure may use any suitable methods for nucleic acid delivery for transformation of a cell, as described herein or as would be known to one of ordinary skill in the art. Such methods include, but are not limited to, direct delivery of DNA such as by ex vivo transfection, by injection, including microinjection); by electroporation; by calcium phosphate precipitation; by using DEAE-dextran followed by polyethylene glycol; by direct sonic loading; by liposome mediated transfection and receptor-mediated transfection; by microprojectile bombardment; by agitation with silicon carbide fibers; by Agrobacterium-mediated transformation; by desiccation/inhibition-mediated DNA uptake, and any combination of such methods. Through the application of techniques such as these, organelle(s), cell(s), tissue(s) or organism(s) may be stably or transiently transformed.

### VI. Gene Editing and CRISPR

The T cell production process of the disclosure includes gene editing of T cells. In some cases, the gene editing occurs in T cells expressing one or more heterologous proteins, including heterologous antigen receptors and/or cytokines, whereas in other cases the gene editing occurs in T cells that do not express the one or more heterologous proteins. In particular embodiments, the T cells that are gene edited are expanded T cells.

In particular cases, one or more endogenous genes of the T cells are modified, such as disrupted in expression where the expression is reduced in part or in full. In specific cases, one or more genes are knocked down or knocked out using processes of the disclosure. In specific cases, multiple genes are knocked down or knocked out in the same step or in different steps as in processes of the disclosure. The genes that are edited in the T cells may be of any kind, but in specific embodiments the genes are genes whose gene products inhibit activity and/or proliferation of T cells. In specific cases, the genes that are edited in the T cells allow T cells to work more effectively in a tumor microenvironment or allow the T cells to be utilized when the individual is in need or one or more glucocorticoids. In specific cases, the genes are one or more of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, TDAG8, CD5, CD7, SLAMF7, CD38, LAG3, TCR, beta2-microglubulin, HLA, CD73, and CD39. In specific embodiments, the TGFBR2 gene is knocked out or knocked down in the T cells. In some embodiments, the NR3C1 is modified, knocked out, or knocked down in the T cells.

In some embodiments, the gene editing is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cast, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). In some cases CpF1 is used instead of the Cas9 protein. In some embodiments, the enzyme is a high fidelity enzyme. The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

### VII. Methods of Treatment

In some embodiments, the T cells produced by the methods of the disclosure are utilized for methods of treatment for an individual in need thereof. Embodiments of the disclosure include methods of treating an individual for cancer, infections of any kind, and/or any immune disorder, as examples. The individual may utilize the treatment method of the disclosure as an initial treatment or after (or with) another treatment, such as following HSCT, for example. The immunotherapy methods may be tailored to the need of an individual with cancer based on the type and/or stage of cancer, and in at least some cases the immunotherapy may be modified during the course of treatment for the individual.

In specific cases, examples of treatment methods are as follows: 1) Adoptive cellular therapy with the produced T cells (ex vivo-expanded or expressing CAR(s) and/or TCR(s)) to treat an individual for post-HSCT viral infection, including with glucocorticoid treatment in some cases; 2) Adoptive cellular therapy with the produced T cells (ex vivo expanded or expressing CAR(s) and/or TCR(s)) to treat cancer patients with any type of hematologic malignancy; (3) Adoptive cellular therapy with the produced T cells (ex vivo expanded or expressing CAR(s) or TCR(s)) to treat cancer patients with any type of solid cancers; and/or (4) Adoptive cellular therapy with the produced T cells (ex vivo-expanded or expressing CAR(s) and/or TCR(s)) to treat patients with infectious diseases or immune disorders.

In some embodiments, the present disclosure provides methods for immunotherapy comprising administering an effective amount of the T cells produced by methods of the present disclosure. In one embodiment, a medical disease or disorder is treated by transfer of T cell populations produced by methods herein and that elicit an immune response. In certain embodiments of the present disclosure, cancer or infection is treated by transfer of a T cell population produced by methods of the disclosure and that elicits an immune response. Provided herein are methods for enhancing therapy with virus-specific T cells when glucocorticoids are also in need of being administered to the patient. Also provided herein are methods for treating or delaying progression of cancer in an individual comprising administering to the individual an effective amount an antigen-specific cell therapy. The present methods may be applied for the treatment of immune disorders, solid cancers, hematologic cancers, and/or viral infections.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

Particular embodiments concern methods of treatment of leukemia. Leukemia is a cancer of the blood or bone marrow and is characterized by an abnormal proliferation (production by multiplication) of blood cells, usually white blood cells (leukocytes). It is part of the broad group of diseases called hematological neoplasms. Leukemia is a broad term covering a spectrum of diseases. Leukemia is clinically and pathologically split into its acute and chronic forms.

In certain embodiments of the present disclosure, immune cells are delivered to an individual in need thereof, such as an individual that has cancer or an infection, including viral infection. The cells then enhance the individual's immune system to attack the respective cancer or pathogenic cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, or more days.

Certain embodiments of the present disclosure provide methods for treating or preventing an immune-mediated disorder. In one embodiment, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or membranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

In yet another embodiment, the subject is the recipient of a transplanted organ or stem cells and immune cells are used to prevent and/or treat rejection. In particular embodiments, the subject has or is at risk of developing graft versus host disease. GVHD is a possible complication of any transplant that uses or contains stem cells from either a related or an unrelated donor. There are two kinds of GVHD, acute and chronic. Acute GVHD appears within the first three months following transplantation. Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Any of the populations of immune cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face Immune cells can be administered prior to transplantation, concurrently with transplantation, or following transplantation. In some embodiments, the immune cells are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of immune cells occurs 3-5 days prior to transplantation.

In some embodiments, the subject can be administered nonmyeloablative lymphodepleting chemotherapy prior to the immune cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m² fludarabine is administered for five days.

In certain embodiments, one or more growth factors that promote the growth and activation of the T cells are administered to the subject concomitantly with the T cells and/or subsequently to the T cells. The growth factor can be any suitable growth factor that promotes the growth and activation of the T cells. Examples of suitable immune cell growth factors include IL-2, IL-7, IL-12, IL-15, IL-18, and IL-21, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

Therapeutically effective amounts of the produced T cells can be administered by a number of routes, including parenteral administration, for example, intravenous, intraperitoneal, intramuscular, intrasternal, intratumoral, intrathecal, intraventricular, through a reservoir, intraarticular injection, or infusion.

The therapeutically effective amount of the produced T cells for use in adoptive cell therapy is that amount that achieves a desired effect in a subject being treated. For instance, this can be the amount of immune cells necessary to inhibit advancement, or to cause regression of an autoimmune or alloimmune disease, or which is capable of relieving symptoms caused by an autoimmune disease, such as pain and inflammation. It can be the amount necessary to relieve symptoms associated with inflammation, such as pain, edema and elevated temperature. It can also be the amount necessary to diminish or prevent rejection of a transplanted organ.

The produced T cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of T cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ T cells/m². In a certain embodiment, the dose used in the treatment of human subjects ranges from about 3.8×10⁹ to about 3.8×10¹⁰ T cells/m². In additional embodiments, a therapeutically effective amount of T cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of T cells is readily determined by one of skill in the art based on the age, weight, sex, and physiological condition of the subject. Effective doses can be extrapolated from dose-response curves derived from in vitro or animal model test systems.

The T cells may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetylsalicylic acid, ibuprofen or naproxen sodium), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B cells); chemotherapeutic agents (e.g., Methotrexate, Treosulfan, Busulfan); irradiation; or chemokines, interleukins or their inhibitors (e.g., BAFF, IL-2, anti-IL-2R, IL-4, JAK kinase inhibitors) can be administered. Such additional pharmaceutical agents can be administered before, during, or after administration of the immune cells, depending on the desired effect. This administration of the cells and the agent can be by the same route or by different routes, and either at the same site or at a different site.

A. Pharmaceutical Compositions

Also provided herein are pharmaceutical compositions and formulations comprising T cells produced by the processes encompassed herein and a pharmaceutically acceptable carrier.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

B. Combination Therapies

In certain embodiments, the compositions and methods of the present embodiments involve a T cell population in combination with at least one additional therapy. The additional therapy may be one or more glucorticoids, radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of one or more small molecule enzymatic inhibitors or one or more anti-metastatic agents. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

A T cell therapy of the disclosure may be administered before, during, after, or in various combinations relative to one or more glucocorticoids. A T cell therapy of the disclosure may be administered before, during, after, or in various combinations relative to an additional cancer therapy, such as immune checkpoint therapy. In any event, the administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the cell therapy and the additional therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an cell therapy is “A” and an additional therapy is “B”:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gamma1I and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein transferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation, and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells.

Antibody-drug conjugates (ADCs) comprise monoclonal antibodies (MAbs) that are covalently linked to cell-killing drugs and may be used in combination therapies. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. Exemplary ADC drugs include ADCETRIS® (brentuximab vedotin) and KADCYLA® (trastuzumab emtansine or T-DM1).

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies include immune adjuvants, e.g., Mycobacterium bovis, Plasmodium falciparum, dinitrochlorobenzene, and aromatic compounds); cytokine therapy, e.g., interferons α, β, and γ, IL-1, GM-CSF, and TNF; gene therapy, e.g., TNF, IL-1, IL-2, and p53; and monoclonal antibodies, e.g., anti-CD20, anti-ganglioside GM2, and anti-p185. It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies. Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody that may be used. Pembrolizumab, also known as MK-3475, Merck 3475, lambrolizumab, KEYTRUDA®, and SCH-900475, is an exemplary anti-PD-1 antibody. CT-011, also known as hBAT or hBAT-1, is also an anti-PD-1 antibody. AMP-224, also known as B7-DCIg, is a PDL2-Fc fusion soluble receptor.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. An exemplary anti-CTLA-4 antibody is ipilimumab (also known as 10D1, MDX-010, MDX-101, and Yervoy®) or antigen binding fragments and variants thereof. In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VIII. Articles of Manufacture or Kits

An article of manufacture or a kit is provided comprising immune cells is also provided herein. An article of manufacture or a kit is provided comprising engineered T cells and/or one or more reagents for generating them. The T cells may be from any source and may be produced by methods encompassed herein or the kit may comprise reagents to generate such engineered T cells. In some embodiments, the T cells have already been modified and may be provided in the kit so that they may be further modified, such as to be gene edited and/or to express one or more heterologous proteins, including antigen receptors and/or heterologous cytokines. In specific embodiments, the T cells have already been modified to express one or more heterologous antigen receptors and/or to be gene edited and may be provided in the kit so that they may be further modified. In specific embodiments, the T cells have already been modified to be gene edited and may be provided in the kit so that they may be further modified to express one or more heterologous antigen receptors. The T cells may have already been modified to be viral-specific, or the kit may comprise one or more reagents that are utilized for engineering the T cells to be viral-specific, such as comprising peptide antigens directed to one or more desired antigens.

In specific embodiments, one or more reagents for generating the T cells are provided in the kit, such as reagents that target a specific gene, reagents that comprise one or more heterologous antigen receptors (or one or more reagents to produce the heterologous antigen receptor(s)), a cytokine transfection or transduction vector or expression construct, viral antigen peptides, or a combination thereof. In general embodiments, the reagents may comprise nucleic acid including DNA or RNA, protein, media, buffers, salts, co-factors, and so forth. In specific cases, the kit comprises one or more CRISPR-associated reagents, including for targeting a specific desired gene.

The article of manufacture or kit can further comprise a package insert comprising instructions for using the immune cells, or cells therefrom, to treat or delay progression of cancer in an individual or to enhance immune function of an individual having cancer. Any of the antigen-specific immune cells described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### EXAMPLES

The following examples are included to demonstrate particular embodiments of the disclosure. It should be appreciated by those of skill in the art that the techniques disclosed in the examples that follow represent techniques discovered by the inventor(s) to function well in the practice of the methods and compositions of the disclosure, and thus can be considered to constitute particular modes for its practice. However, those of skill in the art should, in light of the present disclosure, appreciate that many changes can be made in the specific embodiments that are disclosed and still obtain a like or similar result without departing from the spirit and scope of the disclosure.

### Example 1

### Virus-Specific T Cells

Virus-specific T cells (VSTs) have proven safe and effective for the treatment of life-threatening viral infections such as cytomegalovirus (CMV), Epstein-Ban virus (EBV), adenovirus and BK virus (BKV) after allogeneic hematopoietic stem cell transplant (HSCT) (Bollard and Heslop, 2016). However, after HSCT, many patients receive steroids for the treatment of complications such as graft-versus-host disease (GVHD). Indeed, after HSCT, viral reactivation often follows the use of glucocorticoids (Tong and Worswick, 2015). Glucocorticoids are lymphocytotoxic and induce apoptosis of T cells (Lanza et al., 1996), thereby limiting the clinical efficacy of adoptively infused VSTs. Glucocorticoids exert their potent immunosuppressive effect by binding to the glucocorticoid receptor (GR), a pleiotropic ligand-activated transcription factor (Rhen and Cidlowski, 2005; Kadmiel and Cidlowski, 2013). Therefore, engineering VSTs to render them steroid resistant by deleting the Nuclear Receptor Subfamily 3 Group C Member 1 (NR3C1, the gene encoding for the GR protein) could be a valid strategy for the treatment of patients with life threatening viral infections receiving glucocorticoid therapy.

The present disclosure provides a novel strategy for the production of good manufacturing practice (GMP)-grade multivirus-specific T-cells that have been engineered to silence the expression of the glucocorticoid receptor using CRISPR-Cas9 gene editing.

### Example 2

### Examples of Methods for Multivirus-Specific T Cells

### I. Multivirus Specific T Cell Generation

Peripheral blood mononuclear cells (PBMC) were isolated by Ficoll from buffy coats of seropositive donors. PBMC were then stimulated without selection with virus-specific pepmix (1 ug/ml, JPT) and cultured in RPMI1640+10% human AB serum at 37° C. with 5% CO₂ for 2 hrs. Two hours later a cytokine cocktail was added (IL-7 10 ng/ml, IL-2 50 IU/ml, IL-15 10 ng/ml). Media was changed (RPMI1640+10% human AB serum) and fresh cytokines were added every 2 days for the length of culture.

### II. CRISPR-Cas9 Gene Editing—Small Scale

NR3C1 KO was performed on day 7-10 of expansion of VSTs using ribonucleoprotein (RNP) complex. Two crRNAs targeting exon 2 of the human GR gene were used: crRNA #1 TGAGAAGCGACAGCCAGTGA (SEQ ID NO:1), crRNA #2 GGCCAGACTGGCACCAACGG (SEQ ID NO:2). First, the crRNA+tracrRNA duplex for each crRNA were prepared by incubation at 95° C. for 5 min in a thermocycler at equimolar concentrations. Cas9 protein (IDT) and gRNA (crRNA+tracrRNA combination) were incubated at room temperature for 15 min in a 1:1 ratio (Gundry et al., 2016). The incubation product (gRNA and Cas9 complex) was then used to electroporate 1-2 million VSTs using the Neon transfection system (Thermo Fisher Scientific). Alt-R HiFi Cas9 Nuclease 3-NLS (IDT) was also used in the above-described protocol to form RNP complexes with gRNAs for electroporation into VSTs. Optimized electroporation conditions were 1600 V, 10 ms, 3 pulses, using T buffer. For the off-target studies of gRNA #1 and gRNA #2, Alt-R crRNA and ATTO-labeled tracrRNA (IDT) comprised the guide RNA; RNP complexes were formed with both Alt-R WT and Alt-R HiFi S.p. Cas9 and electroporated into VSTs.

### III. CRISPR-Cas9 Gene Editing-GMP-Compliant, Large Scale

For the large scale CRISPR-Cas9 protocol the 4D Nucleofector™ from Lonza was used. The program that was adopted was EO-115 and the buffer used was P3 buffer for primary cells. As in the small scale CRISPR-Cas9 protocol, the crRNA+tracrRNA duplex for each crRNA was first prepared by incubating them at 95° C. for 5 min in a thermocycler at equimolar concentrations. Cas9 protein (IDT) and gRNA (crRNA+tracrRNA combination) were then incubated at room temperature for 15 min·2.2 uM of Cas9 and 2.4 uM of gRNA was used to electroporate 5×10⁶ cells. For the larger cell doses, reagents were then scaled up by dividing the cell dose of interest by 5×10⁶ and then multiplying this number by the final amount of RNP complex used to electroporate 5×10⁶ cells.

### IV. PCR Gel Electrophoresis

DNA was extracted and purified (QIAamp DNA Blood Mini Kit, Qiagen Inc., Hilden, Germany) from VSTs (control and NR3C1 KO conditions). The Platinum™ SuperFi™ Green PCR Master Mix from Invitrogen was used for PCR amplification using the following PCR primers spanning the Cas9-sgRNA cleavage site of exon 2 of the GR gene:

Exon 2 Forward Primer: GGACTCCAAAGAATCATTAACTCCTGG (SEQ ID NO:3)

Exon 2 Reverse Primer: AATTACCCCAGGGGTGCAGA (SEQ ID NO:4)

DNA bands were separated by polyacrylamide gel electrophoresis prepared with SYBR safe DNA gel stain in 0.5×TBE. Gel images were obtained using GBox machine with GeneSys software (Syngene, Frederick, Md.).

### V. Western Blot

To detect GR protein expression, VSTs were lysed in lysis buffer (IP Lysis Buffer, Pierce Biotechnology Inc., Rockford, Ill.) supplemented with protease inhibitors (Complete Mini, EDTA-free Cocktail tablets, Roche Holding, Basel, Switzerland) and incubated for 30 min on ice. Protein concentrations were determined by the BCA assay (Pierce Biotechnology Inc., Rockford, Ill.). The following primary antibodies were used: Glucocorticoid Receptor (Clone D6H2L) XP Rabbit mAb, and β-actin antibody (Clone 8H10D10), both antibodies were obtained from Cell Signaling Technology. Blots were imaged using the LI-COR Odyssey Infrared Imaging System.

### VI. Flow Cytometry

The following antibodies were used for flow cytometry staining: anti-human CD3 antibody (BV650, clone UCHT1 from BD Bioscience), anti-human CD4 antibody (APC, clone RPA-T4 from Invitrogen), anti-human CD8 antibody (Percp, clone SK1 from Biolegend), anti-human CD62L (BV605, clone DREG-56 from BD Bioscience), anti-human CD45RA (PE-Cy7, clone HI100 from BD Bioscience), anti-human CCR7 (FITC, clone G03H7 from Biolegend). All data were acquired with BD-Fortessa (BD Biosciences) and analyzed with FlowJo software.

### VII. Annexin V Apoptosis Assay

To evaluate the effect of dexamethasone on the viability of VSTs (Cas9 control or NR3C1 KO), the Annexin V apoptosis assay was performed. Cas9 control or NR3C1 KO VSTs were treated with dexamethasone (200 μM, D2915 from Sigma) for 72 hrs, the cells were collected and washed with Annexin V buffer and stained with Annexin V (V500 from BD Bioscience) and Live/dead (efluor 660, Invitrogen) in addition to CD3 (BV650, clone UCHT1 from BD Bioscience), CD4 (APC, RPA-T4 from Invitrogen) and CD8 (Percp, clone SK1 from Biolegend). After gating on T-cells, the proportion of apoptotic (positive for Annexin V) and dead cells (positive for live/dead stain) were determined by flow cytometry.

### VIII. Functional Assays

VSTs were seeded at 100×10³ cells/200 ul/well in the presence of Brefeldin A for 6 h in round bottom 96-well plates with viral pepmix. Cells were then collected and washed with PBS and surface and intracellular staining was performed using BD fixation/permeabilization kit. Cells were stained with live/dead aqua dead cell stain (Thermofisher), anti-human CD3 antibody (BV650, clone UCHT1 from BD bioscience), anti-human CD4 antibody (APC, clone RPA-T4 from Invitrogen), anti-human CD8 antibody (Percp, clone SK1 from Biolegend). CD107a degranulation was measured with anti-human CD107a (LAMP-1) conjugated to Brilliant Violet 785™, Biolegend, San Diego, Calif., USA) was added to the wells at the beginning of the assay. Intracellular cytokine production was determined using antibodies against TNF-α (clone MAb 11 conjugated to Alexa Fluor 700, eBioscience Inc., San Diego, Calif., USA), IFN-γ conjugated to V450 (clone B27, BD Biosciences, San Jose, Calif., USA), and IL-2 (conjugated to PE, clone MQ1-17H12, BD Bioscience) by flow cytometry as previously described (Rouce et al., 2016).

### IX. In Vivo Xenograft Model

To test the engraftment and persistence of NR3C1 KO VSTs in vivo, four groups of NSG mice (10 weeks old female, n=5 per group) were irradiated on day −1 and then injected on day 0 with either 1×10⁶ control VSTs (n=10, 2 groups) or 1×10⁶ NR3C1 KO VSTs (n=10, 2 groups). Half of the mice (one group per condition) were then monitored and the other half were treated with daily dexamethasone 15 mg/kg subcutaneously for 2 weeks. The mice were then sacrificed and the bone marrows harvested for flow cytometry staining. The antibodies used for the in-vivo studies were the following: live dead aqua (Thermofisher), anti-human CD45 antibody (conjugated to Percp, clone HI30, Biolegend), anti-human CD3 CD3 antibody (BV650, clone UCHT1 from BD bioscience), anti-human CD4 antibody (APC, clone RPA-T4 from Invitrogen), anti-human CD8 antibody (Percp, clone SK1 from Biolegend).

### X. Off-Target Identification

The GUIDE-seq method was employed for unbiased discovery of off-target editing events. (Tsai et al., 2015). HEK293 cells that constitutively express the S. pyogenes Cas9 nuclease (“HEK293-Cas9” cells) were used as the source of Cas9. Alt-R® gRNA complexes were formed by combining Alt-R tracrRNA and Alt-R crRNA XT at a 1:1 molar ratio. gRNA complexes were delivered by nucleofection using the Amaxa™ Nucleofector™ 96-well Shuttle™ System (Lonza, Basel, Switzerland). For each nucleofection, 3.5×10⁵ HEK293-Cas9 cells were washed with 1×PBS, resuspended in 20 μL solution SF (Lonza) and combined with 10 μM gRNA together with 0.5 μM GUIDE-seq dsDNA donor fragment. This mixture was transferred into one well of a Nucleocuvette™ plate (Lonza) and electroporated using protocol 96-DS-150. DNA was extracted 72 hrs after electroporation using the GeneJET Genomic DNA purification kit (Thermo Fisher Scientific). NGS library preparation, sequencing, and operation of the GUIDE-seq software was performed as previously described (Tsai et al., 2016), with the additional inclusion of the Needleman-Wunch alignment.

XI. Target Enrichment Via rhAmpSeq for Multiplexed PCR

To quantify editing at off-target sites identified with GUIDE-seq, multiplex PCR coupled to amplicon NGS was performed, using rhPCR (PCR executed in the presence of RNaseH2) (Dobosy et al., 2011) with blocked-cleavable primers. Primers were designed by an algorithm (developed by IDT) for primer cross-comparison and selection based on compatibility with other primers in the multiplex. This amplification technology requires that the primer properly hybridizes to a target site before amplification. Mismatches between target and primer prevent unblocking, thereby increasing specificity and eliminating primers dimers. This approach enables efficient production of highly multiplex PCR amplicons in a single tube. For these experiments, gRNA complexes were delivered into HEK293-Cas9 cells as previously described or complexed with Alt-R HiFi Cas9 nuclease v3 to form an active ribonucleoprotein complex (RNP) which was then directly nucleofected into HEK293 cells at 2 μM together with 2 μM Alt-R Cas9 Electroporation Enhancer (IDT). DNA was extracted 48 hrs after electroporation using QuickExtract DNA Extraction Solution (Epicentre). Genomic DNA was extracted in the same way from NK cells for off-target studies comparing WT and HiFi Alt-R Cas9 RNP complexes with editing mediated by optimized delivery conditions, as described previously (Vakulskas et al., 2018). Locus-specific amplification with rh-primers was performed for 10 cycles followed by a 1.3×SPRI bead clean-up. An indexing round of PCR was performed for 18 cycles to incorporate sample-unique P5 and P7 indexes followed by a 1×SPRI bead clean-up and library quantification by qPCR (IDT). PCR amplicons were sequenced on an Illumina MiSeq instrument (v2 chemistry, 150 bp paired end reads) (Illumina, San Diego, Calif., USA). Data were analyzed using a custom-built pipeline. Data were demultiplexed (Picard tools v2.9; https://github.com/broadinstitute/picard); forward and reverse reads were merged into extended amplicons (flash v1.2.11); reads were aligned against the GRCh38 genomic reference (minimap2 v2.12), and were assigned to targets in the multiplex primer pool (bedtools tags v2.25). Reads were re-aligned to the target, favoring alignment choices with INDELs near the Cas9 predicted cut site. At each target, editing was calculated as the percentage of total reads containing an INDEL within a 4 bp window of the cut site.

### XII. Statistics

Two way Anova test or student t-test were used as appropriate to compare quantitative differences (mean±s.d.) between groups; P-values were two-sided and P<0.05 was considered significant. The indicated statistical tests were performed using Prism software (GraphPad version 7.0c). For the confocal microscopy analysis, data sets were analyzed using unpaired t test. Data present mean±95% confidence interval. Images were assembled using ImageJ.

### Example 3

### Generation of NR3C1 Knockout Multivirus Specific T Cells Using CRISPR-CAS9 Technology

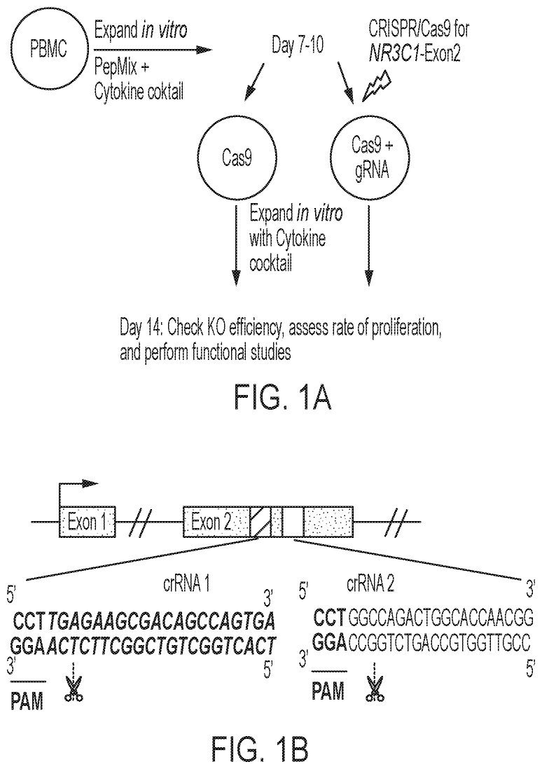

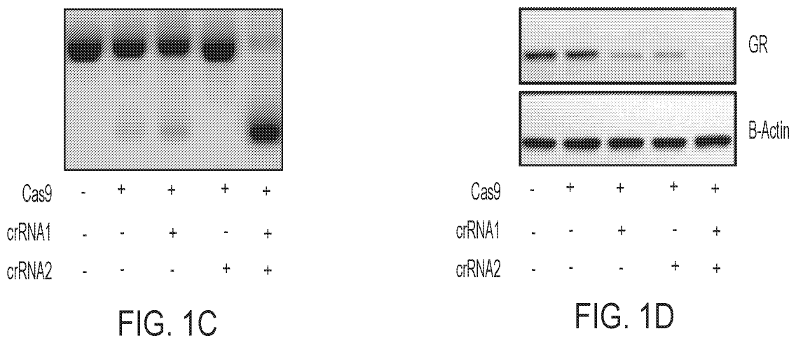

To generate NR3C1 knockout (KO) multivirus specific T-cells (VSTs) against CMV, BKV and adenovirus, peripheral blood mononuclear cells (PBMC) were isolated from seropositive donors and cultured in vitro with pepmixes from the immunodominant proteins of the three viruses in the presence of a cytokine cocktail (IL-2 50 IU/ml, IL-7 10 ng/ml, IL-15 10 ng/ml). On day 7-10 of cell expansion, ribonucleoprotein (RNP) mediated CRISPR-Cas9 gene editing was used to silence NR3C1 in T-cells (FIG. 1A, 1B). Two crRNA sequences were used to target exon 2 of the human NR3C1 gene located on chromosome 5 (FIG. 1B). NR3C1 was efficiently silenced as determined by PCR (FIG. 1C) and western blot analysis (FIG. 1D).

### Example 4

### Silencing Gr Protects Multivirus-Specific T Cells from the Lymphocytotoxic Effect of Glucocorticoids without Altering their Phenotype or Function

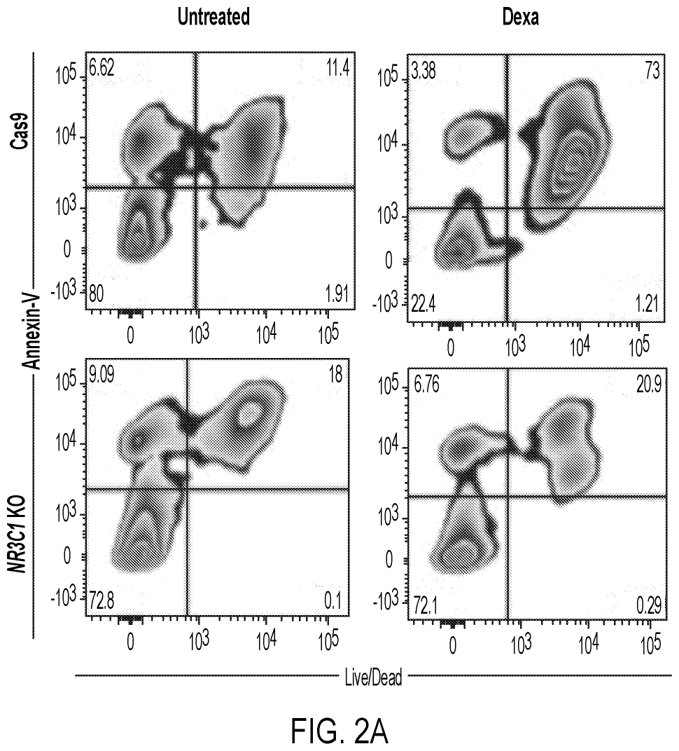

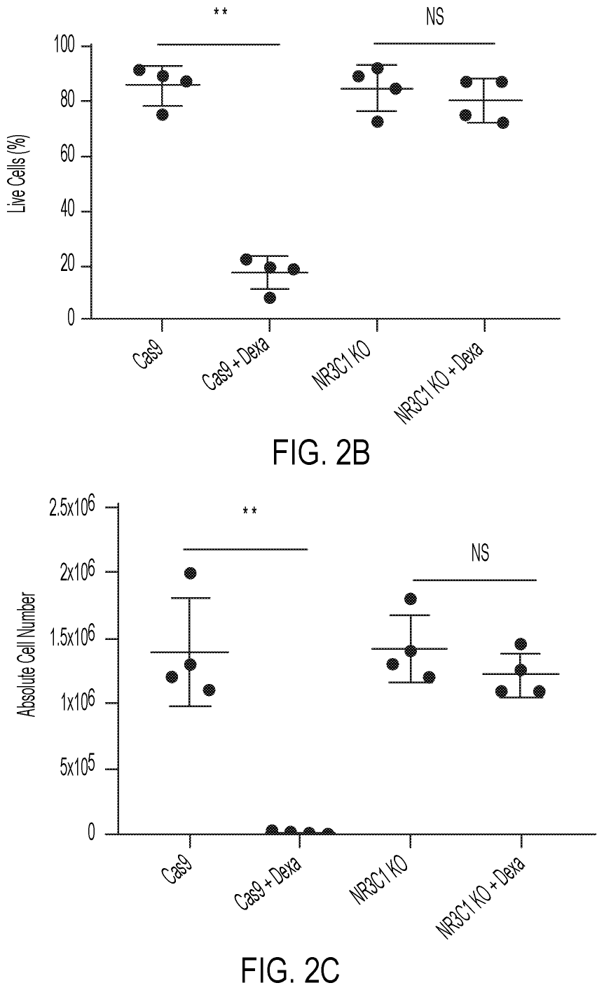

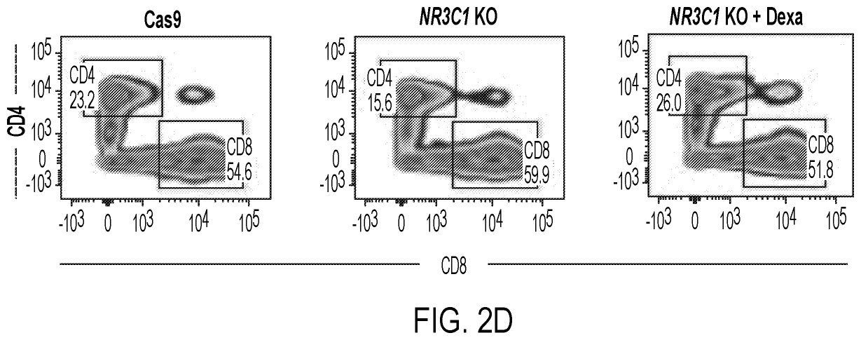

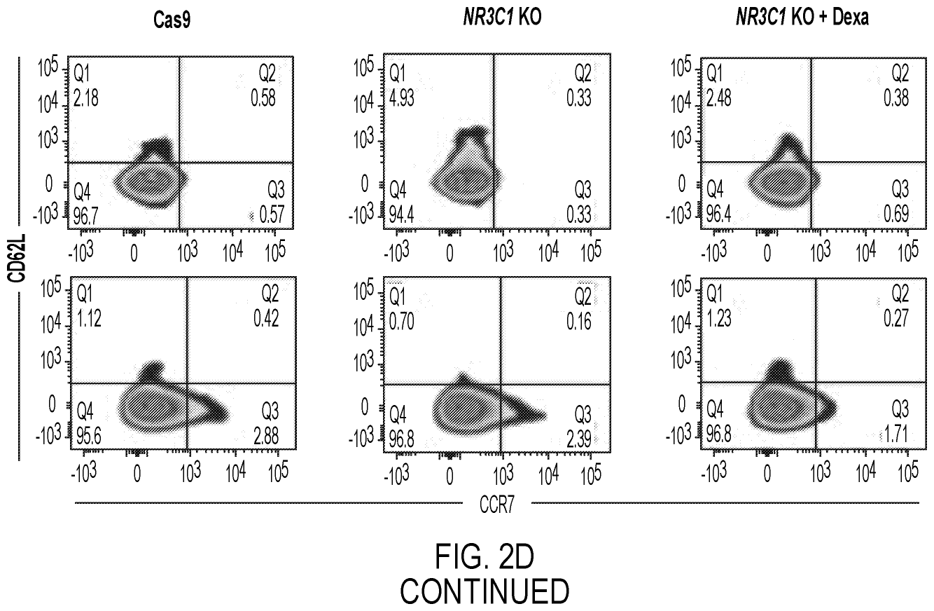

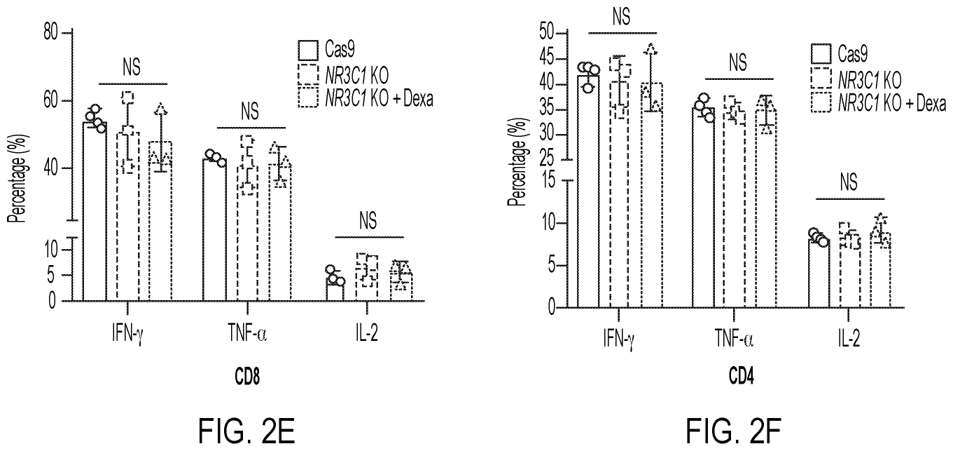

It was determined if silencing the GR protects multivirus specific T cells from the lymphocytotoxic effect of glucocorticoids. NR3C1 KO VSTs or control VSTs (defined in this work as VSTs electroporated with Cas9 alone) were cultured with 200 μM of dexamethasone (Dexa) for 72 hours and their viability was assessed using the annexin V apoptosis assay. At the end of culture, the majority of control VST cells were either apoptotic or dead, while NR3C1 KO cells remained viable, confirming that NR3C1 KO VSTs are resistant to the lymphocytotoxic effect of glucocorticoids (FIGS. 2A-2C).

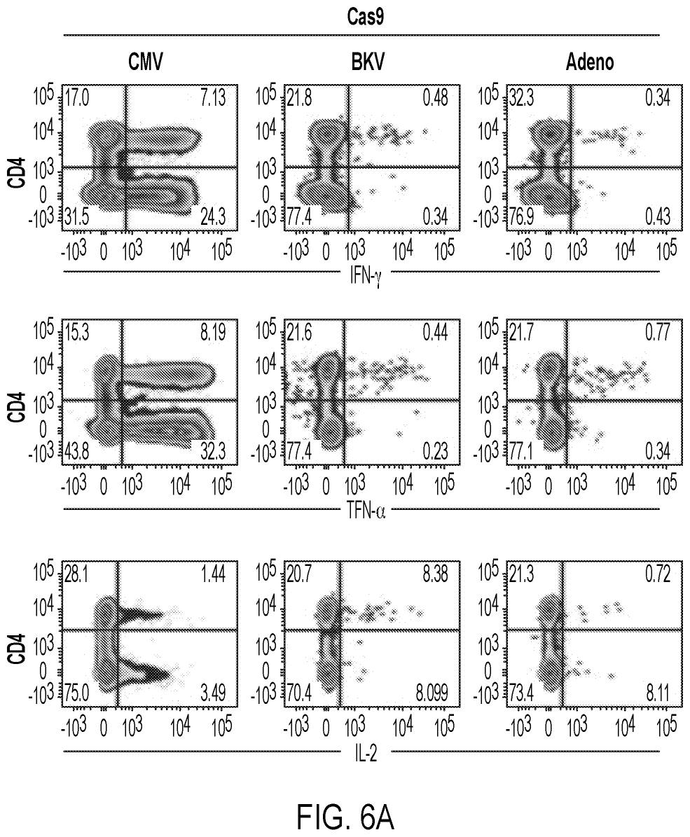

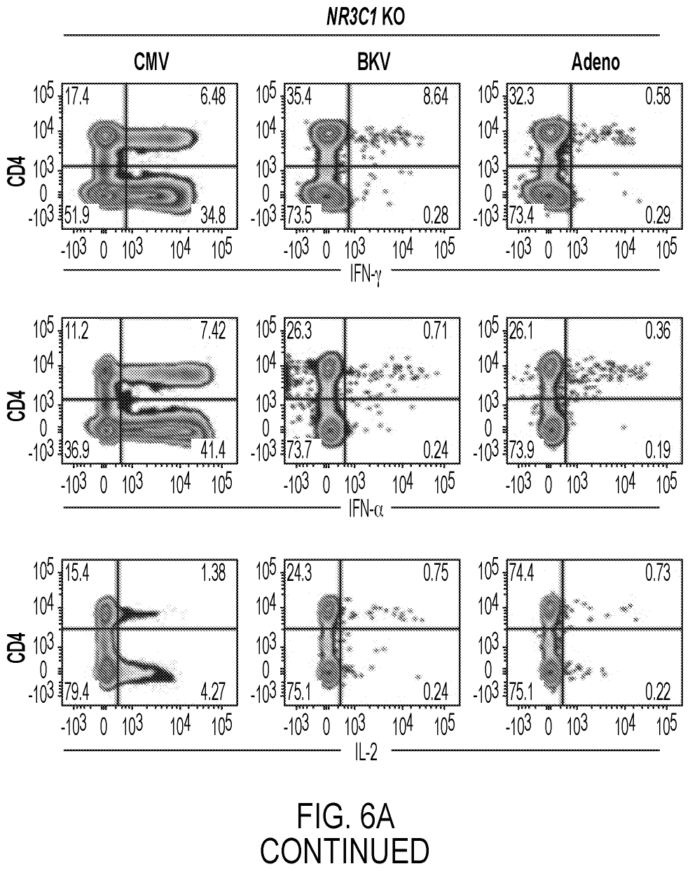

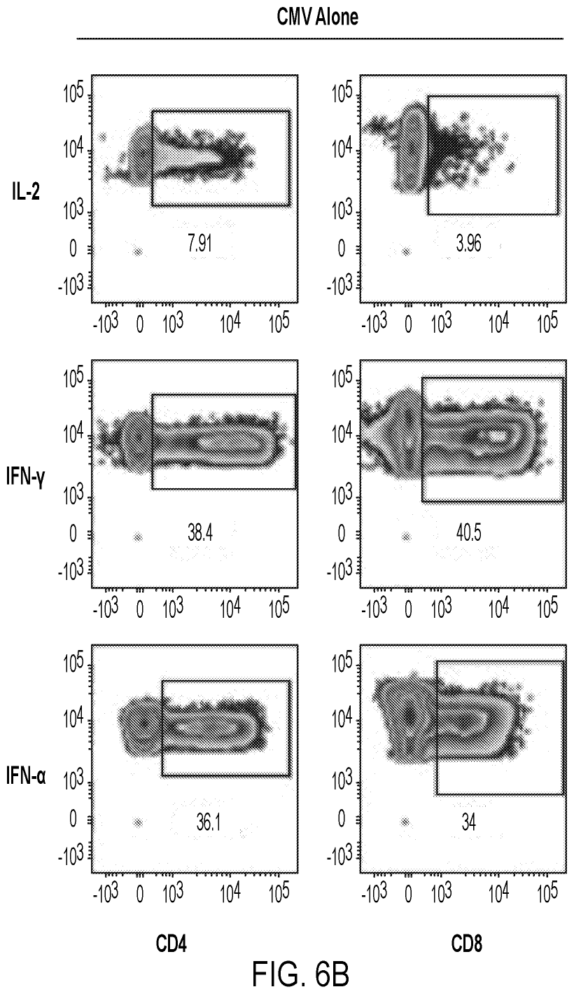

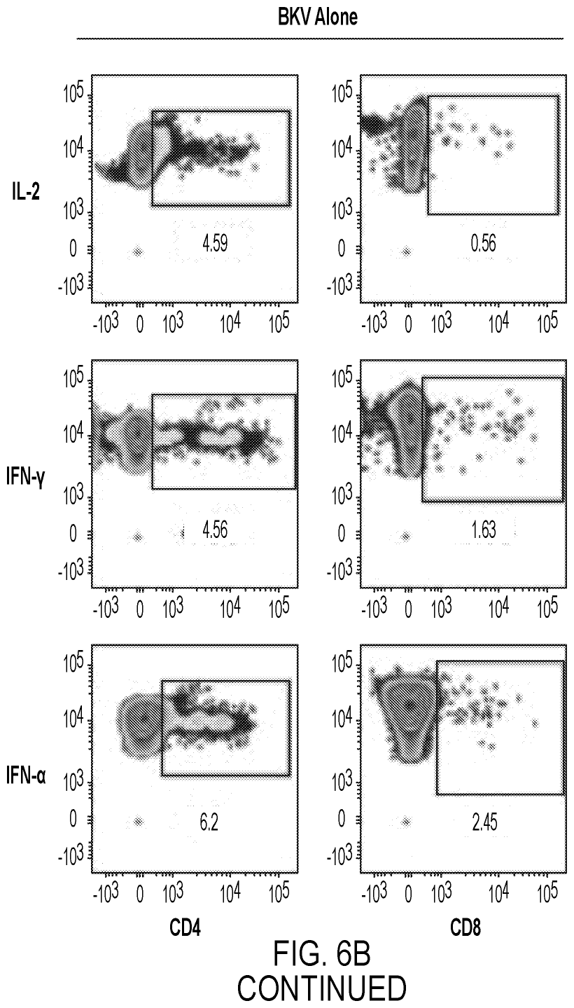

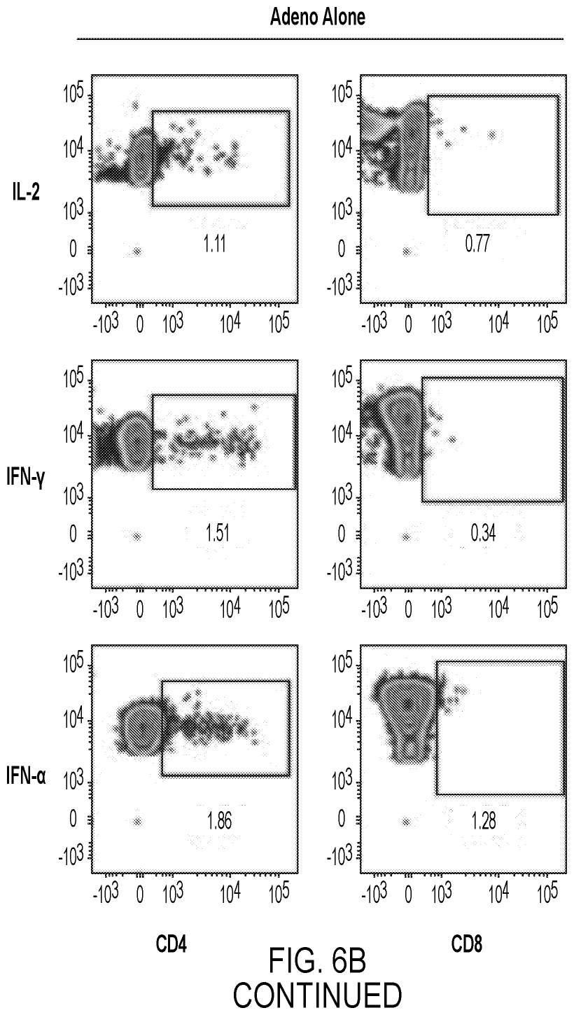

Next, it was examined whether silencing the GR affects the phenotype or function of the multivirus-specific CD4+ and CD8+ T cells. How cytometry analysis confirmed that compared with control VSTs, NR3C1 KO did not affect the distribution of CD4+ and CD8+ or the maturational profile of VSTs (FIG. 2D). NR3C1 KO VSTs had a comparable effector function to control VSTs as measured by production of IFN-γ, TNF-α or IL-2 in response to ex vivo stimulation with viral antigens (FIGS. 2E, 2F and FIGS. 6A, 6B). Furthermore, culture of NR3C1 KO VSTs in the presence of dexamethasone did not affect their effector function against the relevant viral antigens (FIGS. 2E, 2F). These findings confirm that CRISPR-Cas9-mediated knockout of NR3C1 in VSTs protects them from steroid-induced lymphocytotoxicity while preserving their phenotype, function and specificity.

### Example 5

### CRISPR-CAS9 Modified NR3C1 KO VSTS are Resistant to Glucocorticoids in an In Vivo Xenograft Model

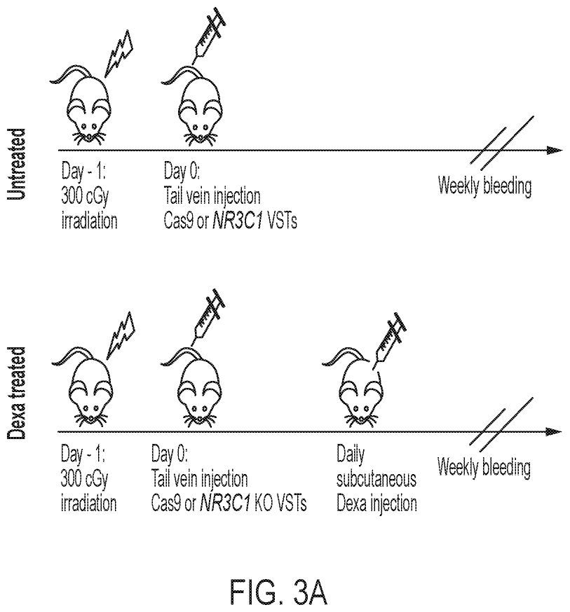

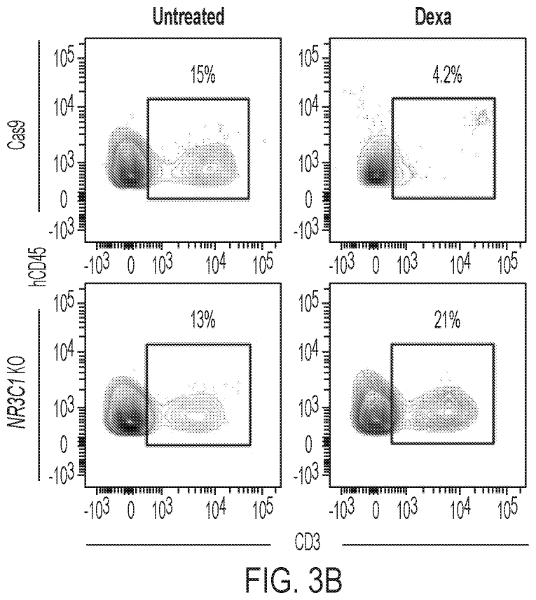

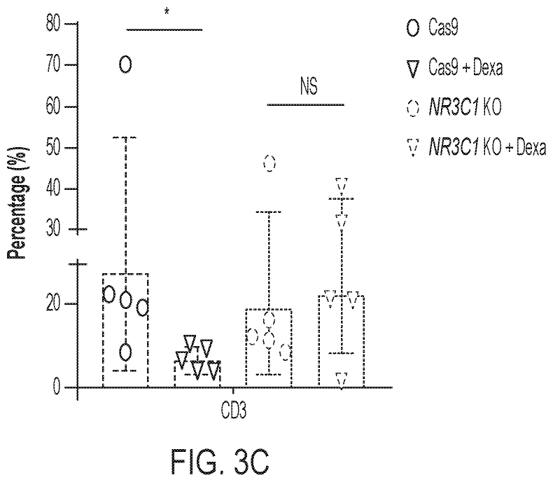

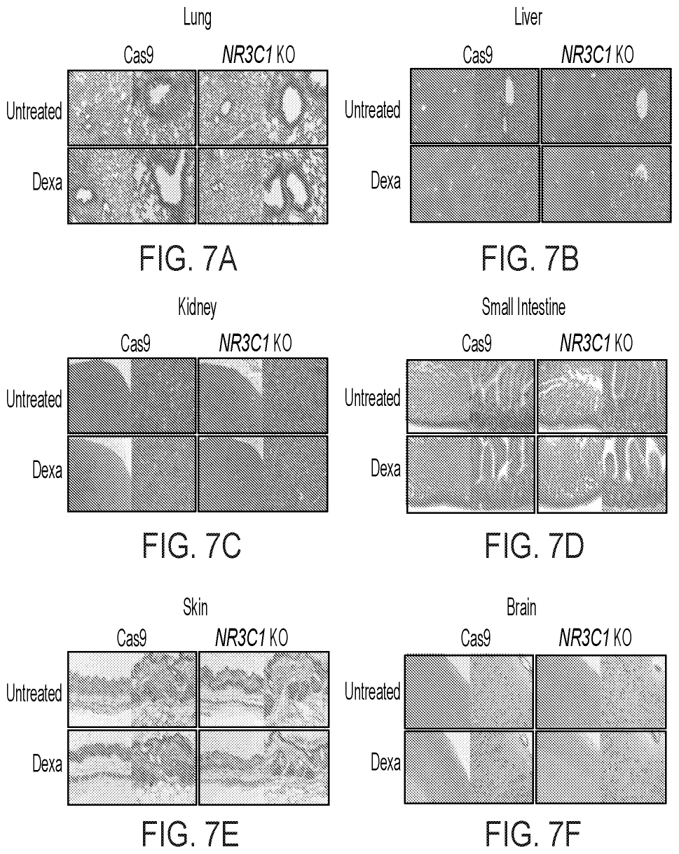

The in vivo persistence of NR3C1 KO VSTs and their resistance to systemic dexamethasone therapy was tested using an immunodeficient mouse model. NSG mice were sublethally irradiated on day −1 and on day 0 they received either 1×10⁶ control VSTs electroporated with Cas9 alone (n=10) or 1×10⁶NR3C1 KO VSTs (n=10). Five mice in each group were then treated with daily dexamethasone 15 mg/kg subcutaneously for 2 weeks and the remaining mice in each group were observed (FIG. 3A). At the end of treatment, the animals were sacrificed and their bone marrow harvested for T-cell enumeration by flow cytometry. In the animals that received control VSTs, as expected, human CD3+ T-cells could only be detected in the group that did not receive dexamethasone (FIGS. 3B, 3C). In contrast, in the animals that received NR3C1 KO VSTs, human T-cells were present at high frequencies (and in comparable numbers) in all animals, irrespective of whether they were treated with dexamethasone or not (FIGS. 3B, 3C). Furthermore human T cell frequencies in mice that received the NR3C1 KO VSTs was similar to that seen in animals treated with unmodified VSTs in the absence of dexamethasone (FIGS. 3B, 3C). No evidence of toxicity including graft-versus-host disease was observed on necropsies in any of the experimental groups (FIG. 7A-7F).

### Example 6

### Analysis of CRISPR-CAS9 Off-Target Cleavage Events in Multivirus Specific T Cells

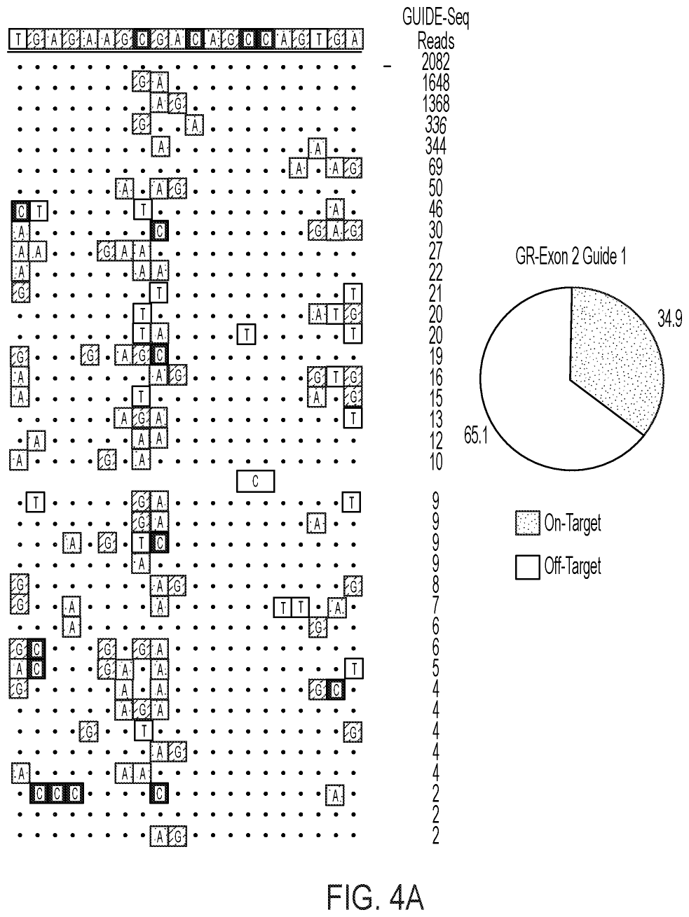

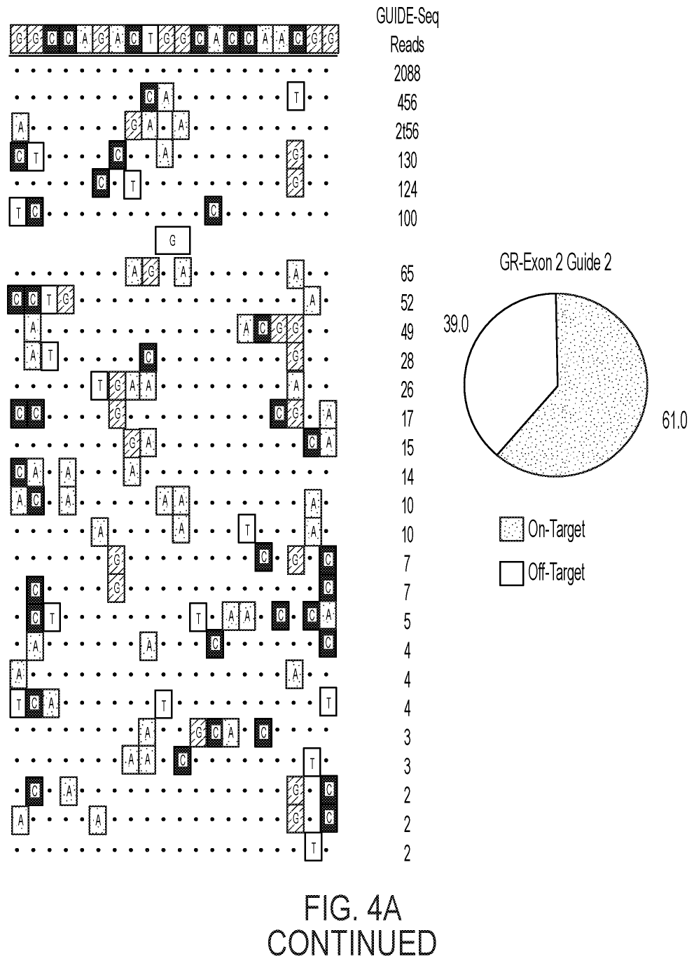

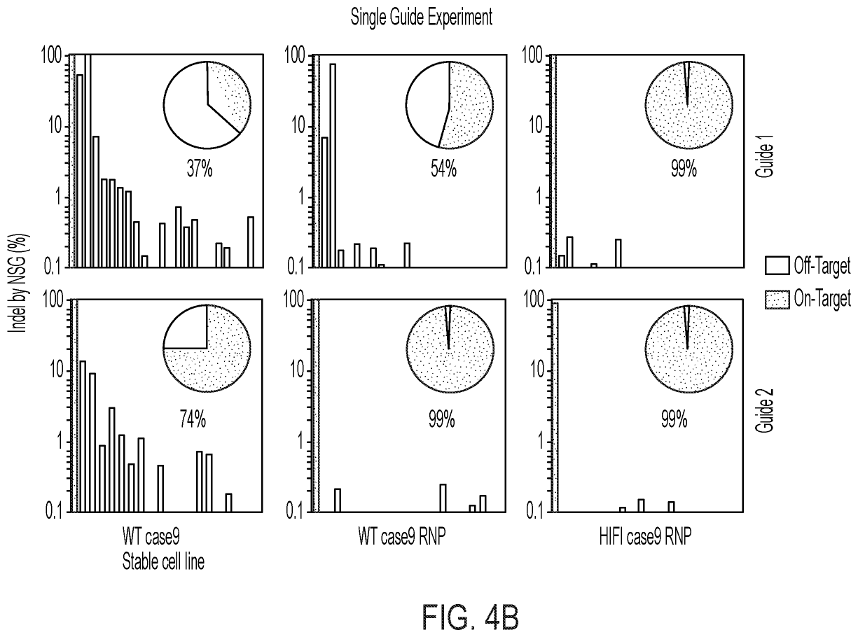

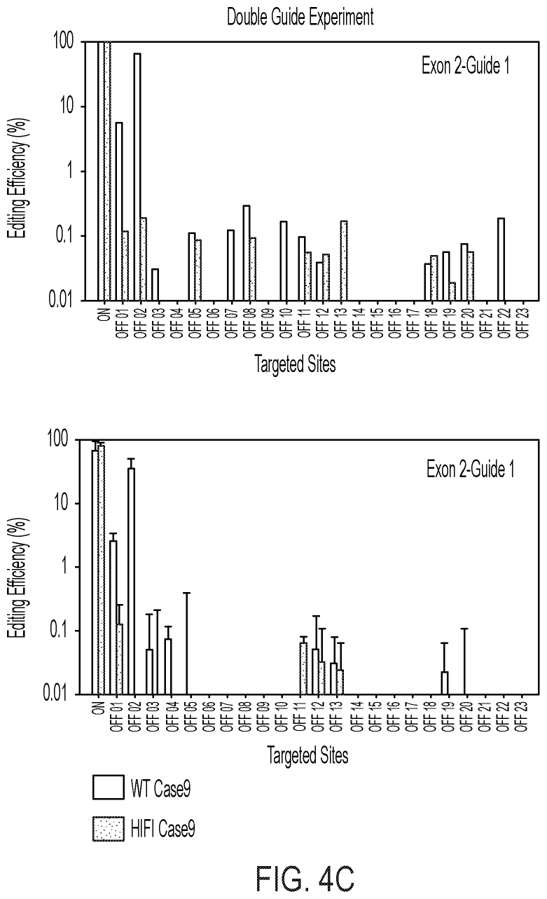

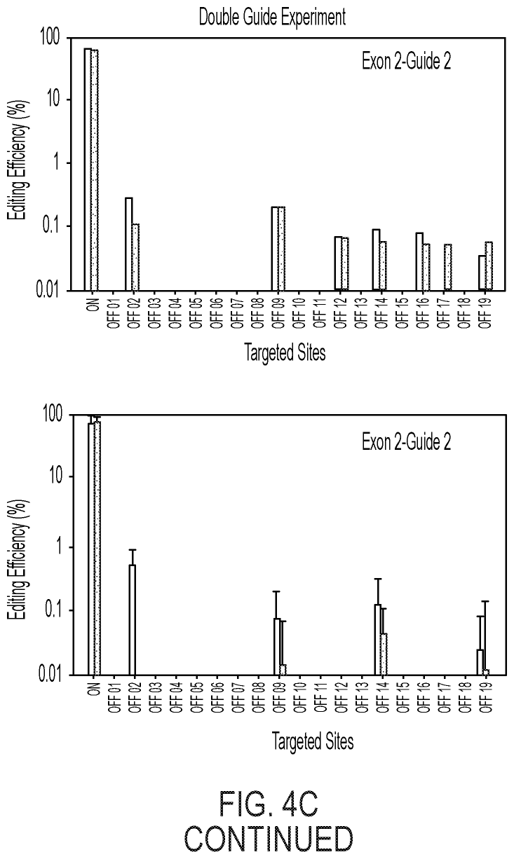

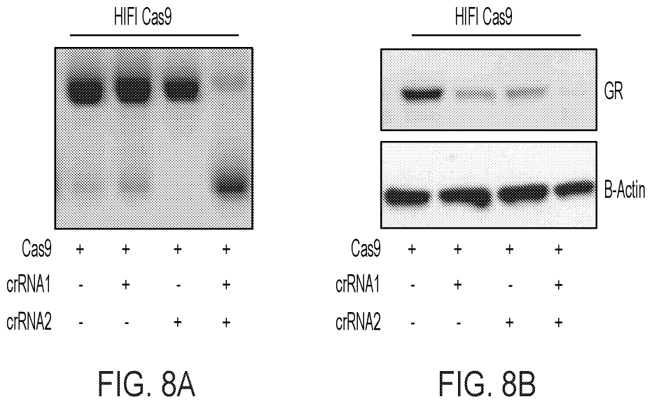

Off-target genome editing is a potential hurdle for the translation of the CRISPR gene editing approach to the clinic. To evaluate the landscape of genome-wide off-target effects for the specific NR3C1 crRNAs used herein, experimental off-target validation was performed using GUIDE-seq and rhAmpSeq™ technologies (Integrated DNA Technologies [IDT]). GUIDE-seq experiments were performed using HEK293 cells that constitutively express S.p Cas9 nuclease paired with highly-modified synthetic gRNAs. The previously published methods⁸ were otherwise followed to identify the off-target sites associated with each crRNA that have the highest potential to be edited (FIG. 4A). Using the rhAmpSeq system, a multiplexed targeted amplicon sequencing technology, the sites identified by GUIDE-seq were focused on to more comprehensively investigate the off-target gene editing events in VSTs electroporated with RNP complexes targeting the NR3C1 locus. Cells treated with wild type S.p. Cas9 protein had a low frequency of off-target editing events with either crRNA1, crRNA2 (FIG. 4B) or with the combination of both crRNAs (FIG. 4C). The use of a high fidelity Cas9 (Alt-R HiFi Cas9 v3, IDT) protein, a Cas9 variant that displays superior on- to off-target ratio when delivered in RNP format11, resulted in efficient KO (FIGS. 8A, 8B) and further reduced the incidence of off-target events to <0.5% (FIG. 4B,C). The high on-target editing activity associated with minimal off-target events supports the translation of this approach to the clinic.

### Example 7

### Clinical Scale Manufacture of GMP Grade NR3C1 KO Multivirus Specific VSTS

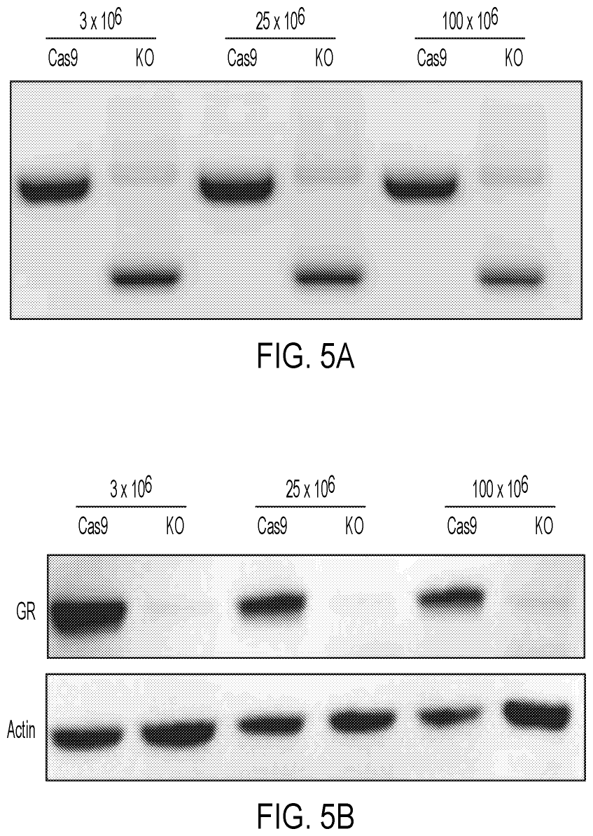

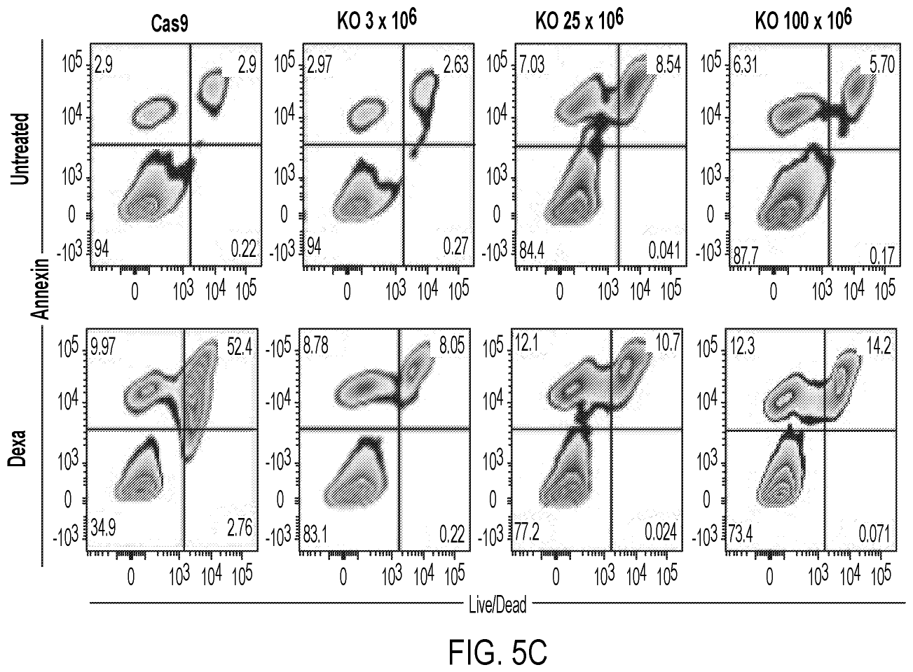

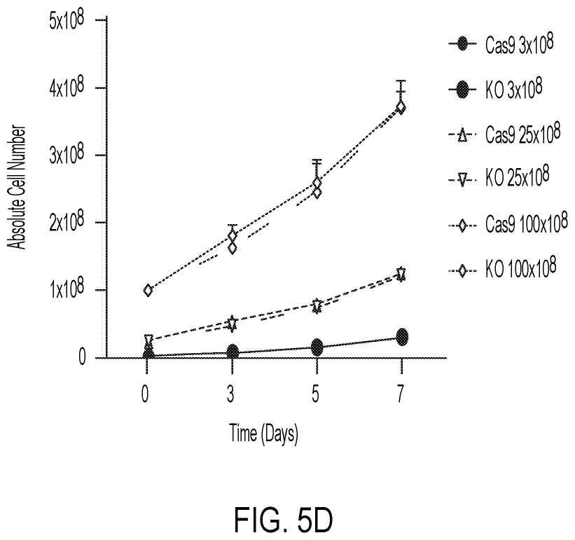

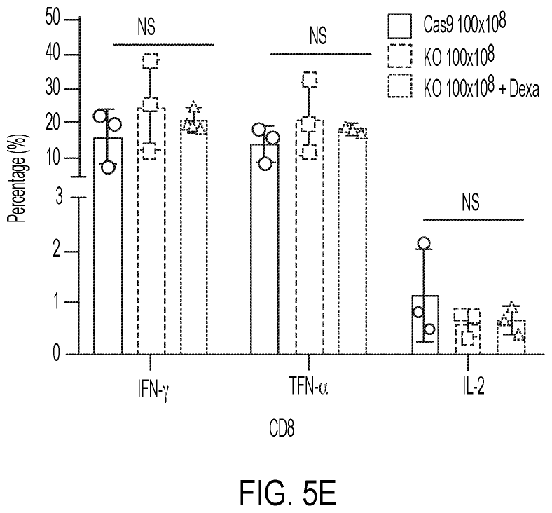

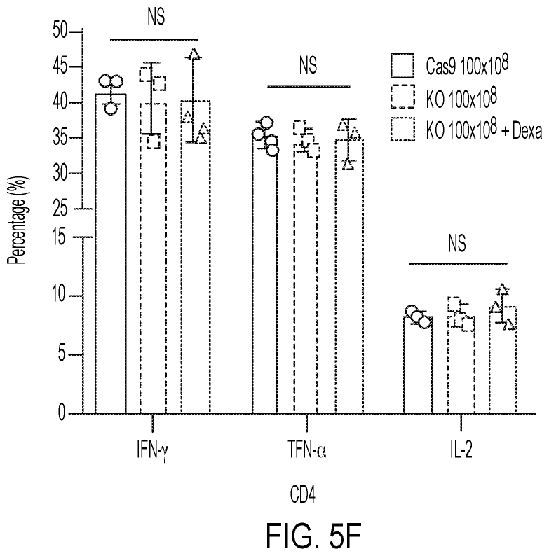

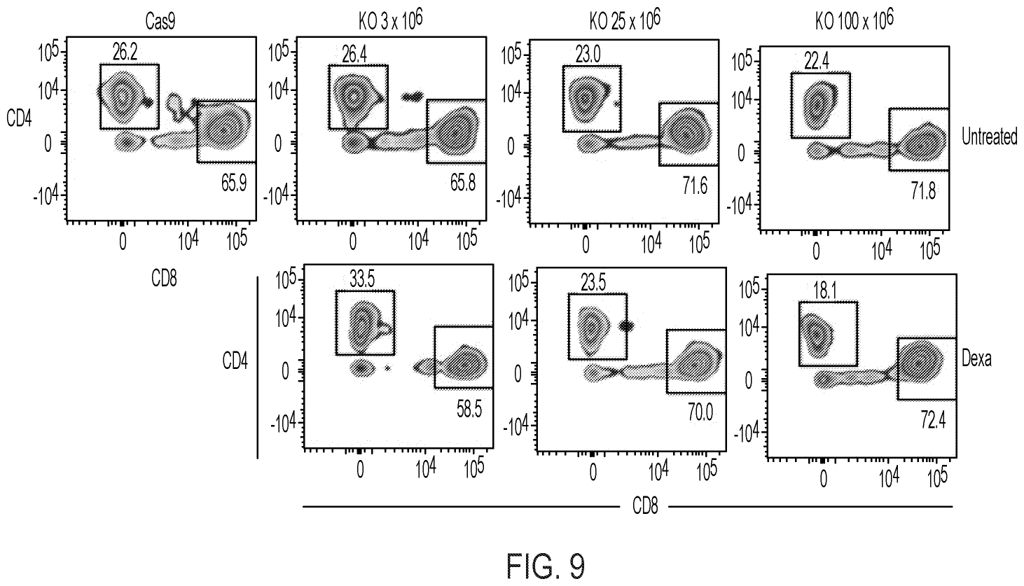

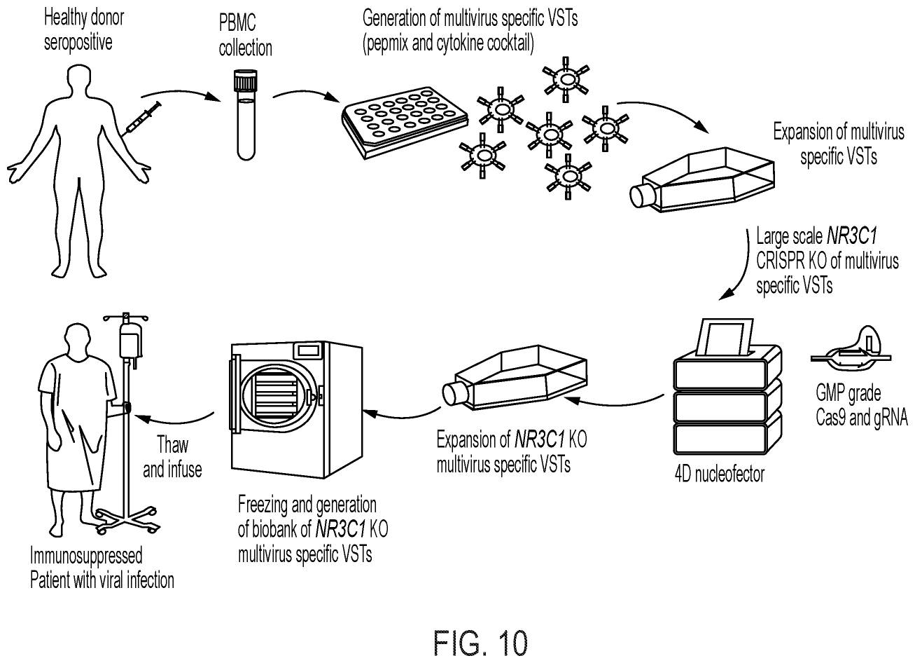

Methods were developed to produce clinically relevant numbers of GMP-compliant NR3C1 KO VSTs. The protocol was optimized using GMP-compliant material, including clinical grade gRNA (Synthego, Calif. USA) and SpyFi Cas9 (the GMP version of HiFi Cas9, Aldevron, N. Dak., USA). The scale-up protocol is detailed in the methods section. Briefly T-cells were expanded using GLP-grade pepmixes from the immunodominant proteins of CMV, BKV and adenovirus. On day 7 to 10 of expansion, different VST cell numbers (3×10⁶, 25×10⁶ and 100×10⁶) were electroporated in the presence of Cas9 and gRNA using the Lonza 4D nucleofector. The cells were expanded for another 4 to 7 days and then harvested for functional studies. The KO efficiency was high and equivalent at both the DNA and protein levels at the three dose levels tested (FIGS. 5A, 5B). Functional studies revealed that at all dose levels tested, the viability and proliferation capacity of GMP grade NR3C1 KO VSTs, even when cultured with dexamethasone, was similar to that of controls (FIGS. 5C, 5D). Furthermore the NR3C1 KO VSTs generated with large scale nucleofection maintained similar distribution of CD4 and CD8 compartments compared to control VSTs, even after treatment with dexamethasone (FIG. 9). Moreover, large scale NR3C1 KO VSTs preserved their effector function and their ability to produce IFN-γ, TNF-α and IL-2 in response to stimulation with the virus pepmix in both the CD8 and CD4 T-cell compartments (FIGS. 5E, 5F). These data prove that scale up of this strategy using GMP-grade materials is technically feasible and attractive for clinical translation. The ultimate goal is to generate a large biobank of these next generation GMP grade NR3C1 KO VSTs to treat viral infections in immunosuppressed patients receiving glucocorticoid therapy (FIG. 10).

### Example 8

### Large Scale GMP Compliant CRISPR-CAS9 Mediated Deletion of the Glucocorticoid Receptor for Use in Multivirus Specific T Cells

Viral reactivation is a common complication after HSCT with significant risks of morbidity and mortality (Ljungman, 2002). Conventional antiviral treatments are either toxic (e.g ganciclovir or foscarnet for CMV) or have limited efficacy (e.g. for adenovirus or BKV). Thus, a number of centers have developed VSTs and have shown the feasibility, safety and efficacy of this approach to treat viral infections post-HSCT (Bollard and Heslop, 2016; Muftuoglu et al., 2018; Tzannou et a., 2017). Viral reactivation is often triggered by the administration of glucorticoids for the management of complications post-HSCT such as GVHD (Tong and Worswick, 2015; Devetten and Vose, 2004), making such patients ineligible for VST therapy. Embodiments encompassed herein encompass novel strategies for the generation of glucocorticoid-resistant multivirus-specific T cells by deleting the NR3C1 gene using CRISPR-Cas9 RNP-mediated gene editing technology. This approach was highly efficient with negligible off-target events. It was shown that NR3C1 KO VSTs are resistant to the lymphocytotoxic effect of glucocorticoids both in vitro and in vivo, while preserving their phenotype and functionality. Finally, methods encompassed herein provide for the GMP-compliant production of NR3C1 KO CMV, BKV and adenovirus multi-specific T-cells for clinical use.

Silencing the GR to render CMV-specific T cells resistant to the effect of glucocorticoids has been performed by others using the TALEN technology (Menger et a., 2015). However CRISPR-Cas9 has a number of advantages over TALEN, including ease of design for genomic targets, easy prediction regarding off-target sites, and the possibility of modifying several genomic sites simultaneously (multiplexing) (Knot et al., 2018; Fellmann et al., 2017; Urnov, 2018). In spite of these advantages, CRISPR-Cas9 carries a number of uncertainties that have complicated its therapeutic translation, including the risk of off-target genome editing (Fu et al., 2013) and challenges in translating this technology safely to the clinic (Nocol et al., 2017; Zhang et al., 2014). Embodiments encompassed herein address these concerns by developing a GMP-compliant protocol for the large-scale expansion and gene editing of multivirus specific T cells. In addition, methods encompassed herein provide a number of steps to assure the safety of the approaches by minimizing the possibility of off-target genetic events. These steps include designing the optimal gRNA based on computational and mathematical predictions and the application of functional experimental off-target cleavage validation assays using GUIDE-seq (Tsai et al., 2015) and rhAmp-Seq (Dobosy et al., 2011) technologies. In addition, an RNP complex was used for Cas9-sg RNA delivery. This approach has been shown to limit off-target events as the RNP degrades rapidly in the cells, limiting its activity (Kim et al., 2014). Finally, GMP-grade high fidelity Cas9 protein (Aldevron) (Jackow et al., 2019) was used to further minimize genome-wide off-target activities to ensure the safety of the methods and compositions encompassed herein.

Adoptive cellular therapy with off-the-shelf HLA-mismatched VSTs is a good platform to introduce CRISPR-Cas9 gene editing to the clinic (Perales et al., 2018; Salas-Mckee et al., 2019). Non-gene edited VSTs have a proven track record of safety as reported in multiple clinical trials (Bollard and Heslop, 2016). They are differentiated mature T-cells with minimal, if any, potential for malignant transformation. Furthermore, they will eventually be rejected on account of the HLA mismatch between the donor and the recipient and in the unlikely event of toxicity, NR3C1 KO VSTs can still be eliminated using antibodies against T-cells such as anti-thymocyte globulin or alemtuzumab (that targets CD52).

In order to translate the methods and compositions encompassed herein to the clinic, validation of the use of GMP-grade viral pepmix and GMP-grade gRNA and high-fidelity Cas 9 protein was performed, and scaled up the transfection protocol to facilitate the generation of clinically relevant numbers of VSTs. It was shown that NR3C1 KO VSTs generated using our GMP-compliant approach have a comparable potency to non-gene edited VSTs, even when cultured in the presence of glucocorticoids. In addition, in an immunodeficient mouse model, the NR3C1 KO VSTs engrafted, could be detected at similar frequencies to non-gene edited VSTs and were resistant to the lymphocytotoxic effect of dexamethasone. The potency of NR3C1 KO VSTs was confirmed in a series of in vitro experiments since a robust mouse model of human viral infection to test the efficacy of adoptively infused VSTs is not readily available.

The methods for the large-scale deletion of the GR can be extended to engineer other cellular therapy products to target cancers where concomitant use of glucocorticoids is often required, such with as brain cancers (Ly and Wen, 2017; Dietrick et al., 2011). In addition it can be applied to engineer regulatory T-cells to allow their co-administration with steroids for the treatment of GVHD (Fisher et al., 2019) or autoimmune disorders (Ellebrecht et al., 2016; Flemming, 2016). Indeed, the methodology to silence the expression of genes using CRISPR-Cas9 and to produce large-scale GMP compliant cells for cell therapy could be used to target a myriad of genes, opening the way for multiple clinical applications.

In summary, methods encompassed herein provide a novel approach for the generation of large numbers of GMP-compliant, steroid-resistant multivirus specific T cells, targeting CMV, BKV and adenovirus, that can be used to treat severe viral infections in immunosuppressed patients, irrespective of whether the patient is receiving glucocorticoid therapy.

### Example 9

### Large Scale NR3C1 Knockout Protocol

This present aspect concerns one example of a large-scale knockout protocol for the gene NR3C1. In this particular case, as an example, the gene editing was performed by CRISPR techniques.

### Examples of Materials

1. gRNA #1 (61 uM, Synthego)
2. gRNA #2 (61 uM, Synthego)

### 3. SpyFY Cas9 (61 uM, Aldevron)

4. Plasmalyte supplemented with HEPES (15 mM) and Mannitol (50 mM)

### 5. Lonza 4D Nucleofactor (Program: Primary P3, CA137, CM137)

6. Culture flasks

Procedure: Below is a Protocol for 100E6 Cells

Step 1: Combine gRNA and cas9 together in the molar ratio of 3:1. Do this separately for all gRNAs.

| | volume | | concentration |
|---|---|---|---|
| gRNA # 1 | 22.50 | ul | 36.6 uM |
| Cas9 | 7.5 | ul | 12.2 uM |
| Plasmalyte | 7.5 | ul | |
| total volume | 5 | ul | |

| | volume | | concentration |
|---|---|---|---|
| gRNA # 2 | 22.5 | ul | 36.6 uM |
| Cas9 | 7.5 | ul | 12.2 uM |
| Plasmalyte | 7.5 | ul | |
| total volume | 5 | ul | |

Mix with pipette, and centrifuge.

Incubate the mixture at room temperature for 20 min

Step 2: Combine the gRNA #1+ Cas9 and gRNA #2+ Cas9 in 1:1 ratio

| | volume | | concentration |
|---|---|---|---|
| gRNA1 + Cas9 | 37.5 | ul | 18.8 uM/6.1 uM |
| gRNA2 + Cas9 | 37.5 | ul | 18.8 uM/6.1 uM |
| total volume | 10 | ul | |

Step 3: Perform electroporation of cells.

Prepare culture flasks with appropriate media prior to electroporation.

Prepare 100E6 cells and re-suspend in 1 ml Plasmalyte just prior to use.

| | volume | concentration |
|---|---|---|
| gRNA#1, 2 + Cas9 | 75 ul | 1 uM/300 ng |
| Cell suspension | 1000 ul | |
| total volume | 1075 ul | |

Use Lonza 4D Nucleofactor system to electroporate using program code CA137 or CM137

Electroporate and add to pre-prepared flasks.

Mix and incubate in 37° C.

After 3 days, check knockout efficiency by PCR and/or western blot, for example.

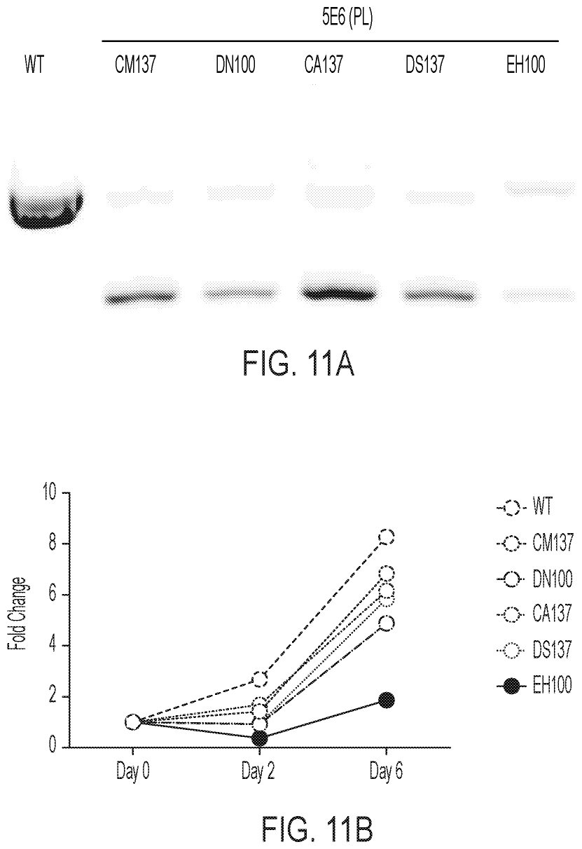

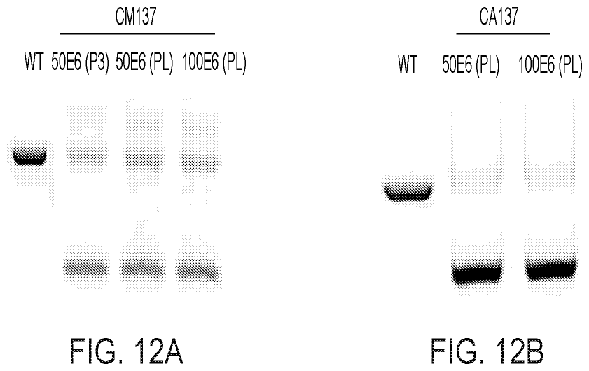

FIG. 11A demonstrates NR3C1 knockout efficiency being tested using PCR for various programs (CM137, DN100, CA137, DS137 and EH100) used for electroporation in Lonza 4D-Nucleofactor™. This was done in small scale, with 5E6 T cells and Plasmalyte (supplemented with HEPES and mannitol, Plasmalyte (PL)). Knockout efficiency using CA137 and CM137 was higher compared to other programs from the manufacturer. The fold expansion of the NR3C1 knocked out cells was tested with the various electroporation programs from the manufacturer (FIG. 11B). Cell viability and expansion rate were higher with CA137 and CM137. Large scale studies (FIGS. 12A-12B) were then performed to test feasibility of these two programs.

In FIG. 12A, NR3C1 knockout efficiency using PCR was tested for the CM137 program used for electroporation in Lonza 4D-Nucleofactor™. This was done in large scale, with 50E6 T cells, using either Lonza buffer (P3) or PL (supplemented with HEPES and mannitol) and with 100E6 T cells using PL (supplemented with HEPES and mannitol). Knockout efficiency and viability was comparable for all conditions. FIG. 12B demonstrates NR3C1 knockout efficiency using the CA137 program utilized for electroporation in Lonza 4D-Nucleofactor™. This was done in large scale, with either 50E6 T cells or 100E6 T cells using PL (supplemented with HEPES and mannitol). Knockout efficiency and viability was comparable for all conditions.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
Ausubel et al., Current Protocols in Molecular Biology, Greene Publishing Associates and John Wiley & Sons, N Y, 1994.
Bollard C M, Heslop H E. T cells for viral infections after allogeneic hematopoietic stem cell transplant. Blood 2016; 127(26): 3331-40.
Chothia et al., The outline structure of the T-cell αβ receptor. EMBO J. 7:3745-3755, 1988.
Cohen et al., Recognition of fresh human tumor by human peripheral blood lymphocytes transduced with a bicistronic retroviral vector encoding a murine anti-p53 TCR. J Immunol. 175:5799-5808, 2005.
Davila et al. CD19 CAR-targeted T cells induce long-term remission and B Cell Aplasia in an immunocompetent mouse model of B cell acute lymphoblastic leukemia. PLoS ONE 8(4): e61338, 2013.
Dietrich J, Rao K, Pastorino S, Kesari S. Corticosteroids in brain cancer patients: benefits and pitfalls. Expert Rev Clin Pharmacol 2011; 4(2): 233-42.
Devetten M P, Vose J M. Graft-versus-host disease: how to translate new insights into new therapeutic strategies. Biol Blood Marrow Transplant 2004; 10(12): 815-25.
Dobosy J R, Rose S D, Beltz K R, et al. RNase H-dependent PCR (rhPCR) improved specificity and single nucleotide polymorphism detection using blocked cleavable primers. BMC Biotechnol 2011; 11: 80.
Ellebrecht C T, Bhoj V G, Nace A, et al. Reengineering chimeric antigen receptor T cells for targeted therapy of autoimmune disease. Science 2016; 353(6295): 179-84.
Fellmann C, Gowen B G, Lin P C, Doudna J A, Corn J E. Cornerstones of CRISPR-Cas in drug discovery and therapy. Nat Rev Drug Discov 2017; 16(2): 89-100.
Fisher S A, Cutler A, Doree C, et al. Mesenchymal stromal cells as treatment or prophylaxis for acute or chronic graft-versus-host disease in haematopoietic stem cell transplant (HSCT) recipients with a haematological condition. Cochrane Database Syst Rev 2019; 1: CD009768.
Flemming A. Autoimmune diseases: CAR-T cells take aim at autoimmunity. Nat Rev Drug Discov 2016; 15(9): 603.
Fu Y, Foden J A, Khayter C, et al. High-frequency off-target mutagenesis induced by CRISPR-Cas nucleases in human cells. Nat Biotechnol 2013; 31(9): 822-6.
Gundry M C, Brunetti L, Lin A, et al. Highly Efficient Genome Editing of Murine and Human Hematopoietic Progenitor Cells by CRISPR/Cas9. Cell Rep 2016; 17(5): 1453-61.
Heemskerk et al. Adoptive cell therapy for patients with melanoma, using tumor-infiltrating lymphocytes genetically engineered t secrete inerleukin-2. Hum Gene Ther. 19:496-510, 2008.
Jackow J, Guo Z, Hansen C, et al. CRISPR/Cas9-based targeted genome editing for correction of recessive dystrophic epidermolysis bullosa using iPS cells. Proc Natl Acad Sci USA 2019. Perales M A, Kebriaei P, Kean L S, Sadelain M. Building a Safer and Faster CAR: Seatbelts, Airbags, and CRISPR. Biol Blood Marrow Transplant 2018; 24(1): 27-31.
Johnson et al. Gene therapy with uman and mouse T-cell receptors mediates cancer regression and targets normal tissues expressing connate antigen. Blood 114:535-46, 2009.
Jores et al., PNAS U.S.A. 87:9138-9142, 1990.
Kabat et al., “Sequences of Proteins of Immunological Interest, US Dept. Health and Human Services, Public Health Service National Institutes of Health, 1991, 5^{th} ed.).
Kadmiel M, Cidlowski J A. Glucocorticoid receptor signaling in health and disease. Trends Pharmacol Sci 2013; 34(9): 518-30.
Kim S, Kim D, Cho S W, Kim J, Kim J S. Highly efficient RNA-guided genome editing in human cells via delivery of purified Cas9 ribonucleoproteins. Genome Res 2014; 24(6): 1012-9.
Knott G J, Doudna J A. CRISPR-Cas guides the future of genetic engineering. Science 2018; 361(6405): 866-9.
Lanza L, Scudeletti M, Puppo F, et al. Prednisone increases apoptosis in in vitro activated human peripheral blood T lymphocytes. Clin Exp Immunol 1996; 103(3): 482-90.
Lefranc et al., IMGT unique numbering for immunoglobulin and T cell receptor variable domains and Ig superfamily V-like domains. Dev. Comp. Immunol. 27:55, 2003.
Li, et al., Directed evolution of human T-cell receptors with picomolar affinities by phage display. Nat Biotechnol. 2005 March; 23(3):349-542005.
Lockey et al., Epstein-Ban virus vaccine development: a lytic and latent protein cocktail. Front. Biosci. 13:5916-27, 2008.
Ljungman P. Prevention and treatment of viral infections in stem cell transplant recipients. Br J Haematol 2002; 118(1): 44-57.
Ly K I, Wen P Y. Clinical Relevance of Steroid Use in Neuro-Oncology. Curr Neurol Neurosci Rep 2017; 17(1): 5.
Menger L, Gouble A, Marzolini M A, et al. TALEN-mediated genetic inactivation of the glucocorticoid receptor in cytomegalovirus-specific T cells. Blood 2015; 126(26): 2781-9.
Muftuoglu M, Olson A, Marin D, et al. Allogeneic BK Virus-Specific T Cells for Progressive Multifocal Leukoencephalopathy. N Engl J Med 2018; 379(15): 1443-51.
Nicol D, Eckstein L, Morrison M, et al. Key challenges in bringing CRISPR-mediated somatic cell therapy into the clinic. Genome Med 2017; 9(1): 85.
Parkhurst et al., Characterization of genetically modified T cell receptors that recognize the CEA:691-699 peptide in the context of HA-A2.1 on human colorectal cancer cells. Clin Cancer Res. 15: 169-180, 2009.
Rhen T, Cidlowski J A. Antiinflammatory action of glucocorticoids—new mechanisms for old drugs. N Engl J Med 2005; 353(16): 1711-23.
Rieder et al., Rhabdovirus evasion of the interferon system. J. Interferon Cytokine Res. (9):499-509, 2009.
Rouce R H, Shaim H, Sekine T, et al. The TGF-beta/SMAD pathway is an important mechanism for NK cell immune evasion in childhood B-acute lymphoblastic leukemia. Leukemia 2016; 30(4): 800-11.
Sadelain et al., The basic principles of chimeric antigen receptor design. Cancer Discov. 3(4): 388-398, 2013.
Salas-Mckee J, Kong W, Gladney W L, et al. CRISPR/Cas9-based genome editing in the era of CAR T cell immunotherapy. Hum Vaccin Immunother 2019; 15(5): 1126-32.
Sambrook et al., Molecular Cloning: A Laboratory Manual, 3rd ed., Cold Spring Harbor Press, Cold Spring Harbor, N.Y. 2001.
Tong L X, Worswick S D. Viral infections in acute graft-versus-host disease: a review of diagnostic and therapeutic approaches. J Am Acad Dermatol 2015; 72(4): 696-702.
Tsai S Q, Zheng Z, Nguyen N T, et al. GUIDE-seq enables genome-wide profiling of off-target cleavage by CRISPR-Cas nucleases. Nat Biotechnol 2015; 33(2):187-97.
Tsai S Q, Topkar V V, Joung J K, Aryee M J. Open-source guideseq software for analysis of GUIDE-seq data. Nat Biotechnol 2016; 34(5): 483.
Tzannou I, Papadopoulou A, Naik S, et al. Off-the-Shelf Virus-Specific T Cells to Treat BK Virus, Human Herpesvirus 6, Cytomegalovirus, Epstein-Ban Virus, and Adenovirus Infections After Allogeneic Hematopoietic Stem-Cell Transplantation. J Clin Oncol 2017; 35(31): 3547-57.
Turtle et al., Engineered T cells for anti-cancer therapy. Curr. Opin. Immunol., 24(5): 633-39, 2012.
Urnov F D. Genome Editing B.C. (Before CRISPR): Lasting Lessons from the “Old Testament”. CRISPR J 2018; 1(1): 34-46.
Vakulskas C A, Dever D P, Rettig G R, et al. A high-fidelity Cas9 mutant delivered as a ribonucleoprotein complex enables efficient gene editing in human hematopoietic stem and progenitor cells. Nat Med 2018; 24(8): 1216-24.
Varela-Rohena et al., Genetic engineering of T cells for adoptive immunotherapy. Immunol Res. 2008; 42(1-3):166-81.
Wu et al., Adoptive T-cell therapy using autologous tumor-infiltrating lymphocytes for metastatic melanoma: current status and future outlook. Cancer, 18(2): 160-75, 2012.
Zhang F, Wen Y, Guo X. CRISPR/Cas9 for genome editing: progress, implications and challenges. Hum Mol Genet 2014; 23(R1): R40-6.
WO2000/14257
WO2012/129514
WO2013/126726
WO2013/166321
WO2013/071154
WO2013/123061
WO2014/031687
WO/2014/055668
U.S. Patent Publication 2002/131960
U.S. Patent Publication 2013/0149337
U.S. Patent Publication 2013/287748
U.S. Pat. No. 5,994,136
U.S. Pat. No. 6,013,516
U.S. Pat. No. 6,410,319
U.S. Pat. No. 6,451,995
U.S. Pat. No. 7,070,995
U.S. Pat. No. 7,265,209
U.S. Pat. No. 7,354,762
U.S. Pat. No. 7,446,179
U.S. Pat. No. 7,446,190
U.S. Pat. No. 7,446,191
U.S. Pat. No. 8,252,592
U.S. Pat. No. 8,324,353
U.S. Pat. No. 8,339,645
U.S. Pat. No. 8,398,282
U.S. Pat. No. 8,479,118
European patent application number EP2537416

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. An in vitro method of producing engineered T cells, comprising the steps of, in any order:
(a) optionally exposing T cells from a mixture of cells to negative or positive selection to enrich the T cells;
(b) expanding for a first time period T cells with an effective amount of one or more of interleukin (IL)-2, IL-4, IL-7, IL-12, IL-15, IL-21 and/or one or more other T-cell tropic cytokines (s);
(c) delivering to the T cells an effective amount of Cas9 or CpF1, and one or more guide RNAs to disrupt expression of one or more endogenous genes in the T cells;
(d) modifying the T cells to express one or more heterologous antigen receptors and/or one or more heterologous cytokines; and
(e) expanding the T cells in the presence of one or more viral antigens and optionally one or more cytokines to produce viral-specific cells.

2. The method of claim 1, further comprising the step of (f) after a second time period, delivering to the T cells an effective amount of Cas9 or CpF1, and one or more guide RNAs to disrupt expression of one or more endogenous genes in the T cells, wherein the one or more guide RNAs in step (c) disrupt expression of a different gene or different genes than the one or more guide RNAs in step (f).

3. The method of claim 1 or 2, wherein the first time period is 30-36 hours.

4. The method of claim 2 or 3, wherein the second time period is 2-3 days.

5. The method of any one of claims 1-4, further comprising the step of modifying the expanded T cells and/or the gene edited T cells to express one or more heterologous antigen receptors and/or one or more heterologous cytokines.

6. The method of claim 2, wherein at any time during the method the cells are expanded in the presence of one or more viral antigens and one or more cytokines to produce viral-specific cells.

7. The method of claim 5, wherein after the modifying step the cells are expanded in the presence of one or more viral antigens and one or more cytokines to produce viral-specific cells.

8. The method of any one of claims 1-7, wherein the tropic cytokine is IL-12, IL-18, and/or IL-21.

9. An in vitro method of producing engineered viral-specific T cells, comprising the steps of:
(a) exposing peripheral blood mononuclear cells to first virus-specific mixture of peptide antigens;
(b) stimulating for a first time period the cells with one or more cytokines specific for stimulation of T cells for the first virus to produce stimulated viral-specific T cells; and
(c) delivering to the stimulated viral-specific T cells an effective amount of Cas9 and one or more guide RNAs to disrupt expression of one or more endogenous genes in the cells, thereby producing gene edited viral-specific T cells.

10. The method of claim 9, further comprising the step of (d) after a second time period, delivering to the gene edited viral-specific T cells an effective amount of Cas9 and one or more guide RNAs to disrupt expression of one or more endogenous genes in the T cells, wherein the one or more guide RNAs in step (c) disrupt expression of different one or more endogenous genes than the one or more guide RNAs in step (d).

11. The method of claim 9 or 10, wherein the first time period is 7-10 days.

12. The method of claim 9, 10, or 11, wherein step (a) is further defined as exposing peripheral blood mononuclear cells to a first virus-specific mixture of peptide antigens, a second virus-specific mixture of peptide antigens, and optionally subsequent nonidentical viral-specific mixtures of peptide antigens.

13. The method of any one of claims 9-12, further comprising the step of transducing or transfecting the stimulated viral-specific T cells and/or the gene edited viral-specific T cells with a vector encoding one or more heterologous antigen receptors and/or one or more heterologous cytokines to produce transgenic gene edited viral-specific T cells.

14. The method of any one of claims 1-13, wherein any of the cells are analyzed.

15. The method of claim 14, wherein the cells are analyzed by functional assay, cytoxicity assay, and/or in vivo activity.

16. The method of claim 14 or 15, wherein the cells are analyzed by flow cytometry, mass cytometry, RNA sequencing, or a combination thereof.

17. The method of any one of claims 1-16, wherein any of the cells are stored.

18. The method of any one of claims 1-17, any of the cells produced by the method are delivered to an individual in need thereof.

19. The method of claim 18, wherein the individual has cancer, an infectious disease, or an immune-related disorder.

20. The method of claim 19, wherein the infectious disease is a viral infection.

21. The method of any one of claims 1-20, wherein at least one of the guide RNAs targets a gene encoding a glucocorticoid receptor.

22. The method of any one of claims 1-21, wherein the one or more gene are selected from the group consisting NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, TDAG8, CD5, CD7, SLAMF7, CD38, LAG3, TCR, beta2-microglubulin, HLA, CD73, CD39, and a combination thereof.

23. The method of claim 22, wherein the gene is NR3C1.

24. The method of any one of claims 9-23, wherein the virus-specific mixture of peptide antigens comprise one or more peptides derived from one or more viruses selected from the group consisting of cytomegalovirus (CMV), Epstein-Ban virus (EBV), adenovirus, BK virus (BKV), SARS-CoV-2 (COVID-19), SARS-CoV, John Cunningham (JC) virus, and a combination thereof.

25. A population of cells produced by the method of any one of claims 1-24.

26. A composition comprising the population of claim 25.

27. The composition of claim 26, formulated in a pharmaceutically acceptable carrier.

28. A method of treating an individual for a medical condition, comprising the step of administering to the individual a therapeutically effective amount of cells produced by the method of any one of claims 1-24.

29. The method of claim 28, wherein the medical condition is viral infection following hematopoietic stem cell transplant.

30. The method of claim 28 or claim 29, wherein the disrupted endogenous gene in the T cells is NR3C1.

31. The method of claim 30, wherein the individual is administered an effective amount of one or more glucocorticoids.

32. The method of claim 28, wherein the medical condition is cancer.

33. The method of claim 32, wherein the cancer comprises a hematological malignancy or a solid tumor.

34. The method of claim 28, wherein the medical condition is infectious disease and/or an immune-related disorder.

35. The method of any one of claims 28-34, wherein the T cells are administered to the individual once or multiple times.

36. The method of claim 35, wherein when the T cells are administered to the individual multiple times, the duration between administrations comprises 1, 2, 3, 4, 5, 6, 7, 8, 9, 10, 11, 12, 13, 14, 15, 16, 17, 18, 19, 20, 21, 22, 23, or 24 hours.

37. The method of claim 35, wherein when the T cells are administered to the individual multiple times, the duration between administrations 1, 2, 3, 4, 5, 6, or 7 days.

38. The method of claim 35, wherein when the T cells are administered to the individual multiple times, the duration between administrations comprises 1, 2, 3, 4, 5, 6, 7, 8, 9, 10, 11, or 12 months.

39. The method of any one of claims 28-38, wherein the individual is administered an effective amount of one or more additional therapies for the medical condition.

40. The method of claim 39, wherein the additional therapy is administered to the individual prior to, during, and/or subsequent to the administration of the T cells.

41. A kit comprising the T cells produced by the method of any one of claims 1-24, and/or one or more reagents to produce the T cells.


SEQUENCE LISTING (8 sequences)

SEQ ID NO: 1 - DNA, 20 bp, Artificial Sequence
  Description of Artificial Sequence Synthetic oligonucleotide
tgagaagcgacagccagtga

SEQ ID NO: 2 - DNA, 20 bp, Artificial Sequence
  Description of Artificial Sequence Synthetic oligonucleotide
ggccagactggcaccaacgg

SEQ ID NO: 3 - DNA, 27 bp, Artificial Sequence
  Description of Artificial Sequence Synthetic primer
ggactccaaagaatcattaactcctgg

SEQ ID NO: 4 - DNA, 20 bp, Artificial Sequence
  Description of Artificial Sequence Synthetic primer
aattaccccaggggtgcaga

SEQ ID NO: 5 - DNA, 23 bp, Artificial Sequence
  Description of Artificial Sequence Synthetic oligonucleotide
ccttgagaagcgacagccagtga

SEQ ID NO: 6 - DNA, 24 bp, Artificial Sequence
  Description of Artificial Sequence Synthetic oligonucleotide
tcactggctgtcggcttctcaagg

SEQ ID NO: 7 - DNA, 23 bp, Artificial Sequence
  Description of Artificial Sequence Synthetic oligonucleotide
cctggccagactggcaccaacgg

SEQ ID NO: 8 - DNA, 23 bp, Artificial Sequence
  Description of Artificial Sequence Synthetic oligonucleotide
ccgttggtgccagtctggccagg